SEQUENCE LISTING (16 sequences)

SEQ ID NO: 1 - protein, 82 aa, Streptococcus pyogenes
DSATHIKFSKRDEDGKELAGATMELRDSSGKTISTWISDGQVKDFYLYPGKYTFVETAAPDGYEVATAITFTVNEQGQVTVN

SEQ ID NO: 2 - protein, 13 aa, Streptococcus pyogenes
AHIVMVDAYKPTK

SEQ ID NO: 3 - protein, 112 aa, Streptococcus pneumoniae
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNDKHYITNEPIPPK

SEQ ID NO: 4 - protein, 12 aa, Streptococcus pneumoniae
KLGDIEFIKVNK

SEQ ID NO: 5 - protein, 122 aa, Clostridium perfringens
MTLKTTVAADGVNGSSEKEALVSFENSKDGVDVKDTIDYKDLVANEKYNLTGKLMHVKDDGSLEEVATKTTEVTAVENGSGQWELDFGNQKLQVGEKYVVFENAESVENLIDTDNNYELDTK

SEQ ID NO: 6 - protein, 22 aa, Clostridium perfringens
QVVKHEDKNDKAQTLIVEKPLE

SEQ ID NO: 7 - protein, 5 aa, synthetic construct
GGGGS

SEQ ID NO: 8 - protein, 6 aa, synthetic construct
GGGSGG

SEQ ID NO: 9 - protein, 115 aa, synthetic construct [sequence omitted]

SEQ ID NO: 10 - protein, 385 aa, synthetic construct
MEFGLSWLFLVAILKGVQCQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSGGAHIVMVDAYKPTKGGSGGGGSGGLVPRGSHMHMDIVLTQSPAIMSASPGEKVTMTCSASSSVSYMNWYKQKSGTSPKRWTYDTSKLASGVPARFSGSGSGTSYSLTISSMEAEDAATYYCQQWSSNPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 129 aa, synthetic construct
QVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSS

SEQ ID NO: 12 - protein, 706 aa, synthetic construct
MEFGLSWLFLVAILKGVQCQVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSSGGSGGDSATHIKFSKRDEDGKELAGATMELRDSSGKTISTWISDGQVKDFYLYPGKYTFVETAAPDGYEVATAITFTVNEQGQVTVNGGGSGGGGSGGLVPRGSHMHMQVQLQQSGAELARPGASVKMSCKASGYTFISYTMHWVKQRPGQGLEWIGYINPRSGYTHYNQKLKDKATLTADKSSSSAYMQLSSLTSEDYAVYYCARSAYYDYDGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 234 aa, synthetic construct
METPAQLLFLLLLWLPESTGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 469 aa, synthetic construct
MEFGLSWLFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKALGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 386 aa, synthetic construct
METPAQLLFLLLLWLPESTGQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSGGKLGDIEFIKVNKGGSGGGGSGGLVPRGSHMHMDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 735 aa, synthetic construct
MEFGLSWLFLVAILKGVQCQVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSSGGSGGKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNDKHYITNEPIPPKGGSGGGGSGGLVPRGSHMHMEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKALGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0109787 A1
(43) Pub. Date: Apr. 23, 2026

(54) PRODUCTION METHOD OF HETEROMULTIMERIC PROTEIN, PROTEIN, NUCLEIC ACID, EXPRESSION VECTOR, TRANSFORMANT, AND PRODUCTION METHOD OF PROTEIN

(71) Applicant: NATIONAL UNIVERSITY CORPORATION YAMAGATA UNIVERSITY, Yamagata-shi, Yamagata (JP)

(72) Inventor: Koki MAKABE, Yonezawa-shi, Yamagata (JP)

(21) Appl. No.: 19/116,921

(22) PCT Filed: Sep. 25, 2023
(86) PCT No.: PCT/JP2023/034748

§ 371 Date: May 29, 2025

(30) Foreign Application Priority Data
Sep. 30, 2022 (JP) 2022-158540

(51) Int. Cl. C07K 16/46; C07K 14/315; C07K 14/33; C07K 16/28; C07K 16/32; C12N 15/85; C12P 21/02

(52) U.S. Cl. CPC C07K 16/468; C07K 14/3156; C07K 14/33; C07K 16/2809; C07K 16/32; C12N 15/85; C12P 21/02; C07K 2317/31; C07K 2317/569; C07K 2319/30; C07K 2319/50

(57) ABSTRACT

Provided is a novel production method with which a heteromultimeric protein such as a bispecific antibody can be produced. The production method of the present disclosure is a method for producing a heteromultimeric protein, and includes a complex formation step and a generation step. The complex formation step is a step of forming a first complex of two proteins by bringing the two proteins into contact with each other. In the complex formation step, the two proteins includes a first protein and a second protein. The first protein includes a first binding tag, a first cleavable domain, and a first domain in this order from the N terminus to the C terminus. The second protein includes a first binding partner capable of binding to the first binding tag, a second cleavable domain, and a second domain in this order from the N terminus to the C terminus.

### TECHNICAL FIELD

The present disclosure relates to a method for producing a heteromultimeric protein, a protein, a nucleic acid, an expression vector, a transformant, and a method for producing a protein.

### BACKGROUND ART

Multispecific antibodies have antigen-binding domains capable of respectively binding to different antigens in one antibody molecule, and those for use in various pharmaceutical applications such as anticancer agents and antihemophilic drugs have been developed by changing target antigens.

However, there is a problem in that the production efficiency of multispecific antibodies is very low and it is difficult to produce multispecific antibodies. For example, bispecific antibodies are composed of two types of heavy chains (H chains) and two types of light chains (L chains). When the bispecific antibodies are produced through expression of two types of H chains and two types of L chains, the number of combinations of two H chains and two L chains in the expressed antibodies is ten, which leads to production of unnecessary nine antibodies in addition to a target bispecific antibody (Patent Literature 1).

### CITATION LIST

### Patent Literature

Patent Literature 1: WO 2013/065708

### SUMMARY OF INVENTION

### Technical Problem

Therefore, it is an object of the present disclosure to provide a novel production method with which a heteromultimeric protein such as a bispecific antibody can be produced.

### Solution to Problem

To achieve the aforementioned object, a production method of the present disclosure (also referred to as a “production method” hereinafter) is a method for producing a heteromultimeric protein, including:
a complex formation step of forming a first complex of two proteins by bringing the two proteins into contact with each other,
   the two proteins including a first protein and a second protein,
   the first protein including a first binding tag, a first cleavable domain, and a first domain in this order from an N terminus to a C terminus,
   the second protein including a first binding partner capable of binding to the first binding tag, a second cleavable domain, and a second domain in this order from an N terminus to a C terminus,
   the first protein and the second protein being capable of forming a dimer via a binding between the first domain and the second domain, and
   the first complex being formed by the first binding tag and the first binding partner binding together and the first domain and the second domain binding together, and
a generation step of generating a heterodimer composed of the first domain and the second domain by cleaving the first cleavable domain and the second cleavable domain in the first complex.

A protein of the present disclosure includes a first binding tag capable of binding to a first binding partner, a first cleavable domain, and a first domain in this order from an N terminus to a C terminus.

A protein of the present disclosure includes a first binding partner capable of binding to a first binding tag, a second cleavable domain, and a second domain in this order from an N terminus to a C terminus.

A protein of the present disclosure includes a second binding tag capable of binding to a second binding partner, a third cleavable domain, and a third domain in this order from an N terminus to a C terminus.

A protein of the present disclosure includes a second binding partner capable of binding to a second binding tag, a fourth cleavable domain, and a fourth domain in this order from an N terminus to a C terminus.

A protein of the present disclosure includes two proteins,
wherein the two proteins include a first protein and a second protein,
the first protein includes a first binding tag, a first cleavable domain, and a first domain in this order from an N terminus to a C terminus,
the second protein includes a first binding partner capable of binding to the first binding tag, a second cleavable domain, and a second domain in this order from an N terminus to a C terminus,
the first protein and the second protein form a dimer via a binding between the first domain and the second domain, and
the first binding tag and the first binding partner bind together.

A nucleic acid of the present disclosure encodes the protein of the present disclosure.

A vector of the present disclosure includes the nucleic acid of the present disclosure.

A transformant of the present disclosure includes the nucleic acid and/or the vector of the present disclosure.

A method for producing a protein according to the present disclosure includes an expression step of expressing the nucleic acid and/or the vector of the present disclosure.

### Advantageous Effects of Invention

With the present disclosure, it is possible to provide a novel production method with which a heteromultimeric protein such as a bispecific antibody can be produced.

### BRIEF DESCRIPTION OF DRAWINGS

FIG. 1 shows schematic diagrams illustrating constituent elements of proteins to be used in a production method of the present disclosure and examples of steps conducted to produce a heterodimeric protein.

FIG. 2 shows schematic diagrams illustrating constituent elements of proteins to be used in a production method of the present disclosure and examples of steps conducted to produce a heterotetrameric protein.

FIG. 3 shows schematic diagrams illustrating constituent elements of proteins to be used in a production method of the present disclosure and examples of steps conducted to produce a heterotetrameric protein.

FIG. 4 is a photograph illustrating a result of SDS-PAGE.

FIG. 5 is a photograph illustrating a result of SDS-PAGE.

FIG. 6 is a graph illustrating a Herceptin elution pattern of size-exclusion chromatography.

FIG. 7 is a graph illustrating a binding of a CD3 antibody light chain and a CD3 antibody heavy chain to CD3-positive cells measured through flow cytometry.

FIG. 8 is a graph illustrating a binding of a Herceptin antibody light chain and a Herceptin antibody heavy chain to HER2-positive cells measured through flow cytometry.

FIG. 9 is a photograph illustrating a result of SDS-PAGE.

FIG. 10 is a photograph illustrating a result of SDS-PAGE.

### DESCRIPTION OF EMBODIMENTS

### Definitions

In this specification, the term “protein” means a peptide polymer composed of unmodified amino acids (natural amino acids), modified amino acids, and/or artificial amino acids. The polymer may be, for example, in the linear form, the branched form, the cyclic form, or the like. The protein can also be referred to as a “peptide” or “polypeptide”.

In this specification, the term “monomeric protein” means a protein in a state of not binding to another protein or not associating with another protein.

In this specification, the term “dimeric protein” means a protein complex formed by two proteins or protein subunits binding to or associating with each other. When the two proteins are the same protein or subunit, the dimeric protein can also be referred to as a “homodimeric protein”. When the two proteins are different proteins or subunits, the dimeric protein can also be referred to as a “heterodimeric protein”.

In this specification, the term “tetrameric protein” means a protein complex formed by four proteins or protein subunits binding to or associating with one another. When the four proteins are the same protein or subunit, the tetrameric protein can also be referred to as a “homotetrameric protein”. When one or more proteins or subunits of the four proteins are different proteins or subunits from the others, the tetrameric protein can also be referred to as a “heterotetrameric protein”.

In this specification, the term “multimeric protein” means a protein complex formed by two or more proteins or protein subunits binding to or associating with each other.

In this specification, the term “binding tag” means a polypeptide or substance capable of specifically binding to another molecule.

In this specification, the term “binding partner” means a polypeptide or substance capable of specifically binding to the binding tag.

In this specification, the term “domain” means a region in a “protein”, “polypeptide”, and/or “peptide” considered as one unit in terms of its three-dimensional structure or function.

In this specification, the term “cleavable domain” means a domain composed of a peptide that can be cleaved into two or more regions through cleavage or degradation caused by self-cleavage activity or a separate substance having cleavage activity. Examples of the separate substances having cleavage activity include proteases, peptidases, altered inteins, and the like.

In this specification, the term “antibody” means a protein that includes one or more polypeptides each substantially or partially encoded by an immunoglobulin gene or immunoglobulin gene fragment. The immunoglobulin gene includes, for example, a gene that encodes a constant region such as κ, λ, α (including α1 and α2), γ (including γ1, γ2, γ3, and γ4), δ, ε, or μ, and a gene that can encode innumerable immunoglobulin variable regions such as the V region, the D region, and the J region. The antibody includes, for example, a heavy chain and a light chain. The light chain includes κ and λ, which form a κ chain and a λ chain, respectively. The heavy chain includes γ, μ, α, δ, or ε, which form IgG, IgM, IgA, IgD, and IgE of immunoglobulin classes, respectively. The antibody may be a structural unit of a typical immunoglobulin (antibody) composed of a tetramer. In this case, the antibody is composed of two identical polypeptide chain pairs, and each pair is composed of one light chain (about 25 kDa) and one heavy chain (about 50 to 70 kDa). In addition, the N terminus of each chain defines a variable region that is mainly involved in antigen recognition and that is composed of about 100 to 110 or more amino acids.

In this specification, the term “antigen-binding fragment” means a polypeptide that is a part or portion of an antibody and that includes an antigen-binding site. The antigen-binding fragment can be obtained by chemically or enzymatically processing an antibody. The antigen-binding fragment can also be obtained through a recombination technique. Examples of the antigen-binding fragment include Fab, Fab′, F(ab′)₂, Fc, and/or an Fv fragment, and derivatives thereof.

In this specification, the term “purification” means identification and separation of a substance, collection of a substance from components in a natural state, a state in which a substance is identified and separated, and/or a state in which a substance is collected from components in a natural state. The “purification” can be achieved by, for example, undergoing at least one purification step. The purification can also be referred to as “isolation”.

In this specification, the term “separation” means separation of a target substance from a substance containing the target substance, and/or a state in which a target substance is separated from a substance containing the target substance. The separation can also be referred to as “liberation”.

In this specification, the term “nucleic acid” means a polymer of deoxyribonucleotides (DNAs), ribonucleotides (RNAs), and/or altered nucleotides. In this specification, when the term “nucleic acid” is used in combination with a specific protein, the “nucleic acid” means a polymer of nucleotides encoding the amino acid sequence of the protein. Examples of the nucleic acid include genome DNA, cDNA, mRNA, and the like. The nucleic acid may be, for example, a single-stranded nucleic acid, a double-stranded nucleic acid, or the like. The nucleic acid is interchangeable with “polynucleotide” or “nucleic acid molecule”.

In this specification, the term “host” means a cell and/or an individual into which an exogenous nucleic acid is to be introduced. When the host is a cell, the host can also be referred to as a “host cell”.

In this specification, the terms “vector” and “expression vector” mean a recombinant plasmid or virus that includes a nucleic acid to be delivered to a host or host cell in vitro or in vivo. Examples of the “vector” and the “expression vector” include viral vectors and non-viral vectors.

In this specification, the term “transformant” means a host into which an exogenous nucleic acid has been introduced.

In this specification, the origin of the protein, polypeptide, or peptide is not particularly limited and is any animal. The animal is, for example, a human or a non-human animal. Examples of the non-human animal include mammals such as a mouse, a rat, a rabbit, a dog, a cat, a cow, a horse, a pig, a monkey, a dolphin, and a sea lion.

Although the following describes examples of the present disclosure, the present disclosure is not limited to the following examples and the like, and can be implemented with any modifications. Also, each description in the present disclosure and embodiments can be applied to other descriptions unless otherwise stated. Note that the expression “to” as used herein means that the numerical or physical values before and after “to” are included. In addition, the expression “A and/or B” as used herein encompasses “only A”, “only B”, and “both A and B”.

### <Method for Producing Heteromultimeric Protein>

An aspect of the present disclosure provides a method for producing a heteromultimeric protein. The production method of the present disclosure includes:
a complex formation step of forming a first complex of two proteins by bringing the two proteins into contact with each other,
   the two proteins including a first protein and a second protein,
   the first protein including a first binding tag, a first cleavable domain, and a first domain in this order from an N terminus to a C terminus,
   the second protein including a first binding partner capable of binding to the first binding tag, a second cleavable domain, and a second domain in this order from an N terminus to a C terminus,
   the first protein and the second protein being capable of forming a dimer via a binding between the first domain and the second domain, and
   the first complex being formed by the first binding tag and the first binding partner binding together and the first domain and the second domain binding together, and
a generation step of generating a heterodimer composed of the first domain and the second domain by cleaving the first cleavable domain and the second cleavable domain in the first complex.

The inventors of the present invention came up with an idea that a dimer composed of two proteins can be efficiently produced by adding a specific binding tag and a specific binding partner to the respective proteins for forming a dimer, and forming a complex through a binding between the binding tag and the binding partner. Then, as a result of extensive research, the inventors of the present invention found that, when the two proteins used are each a protein to which a cleavable domain and the binding tag or the binding partner have been added, a heterodimeric protein is obtained by cleaving the cleavable domains after the formation of the complex, and thus achieved the present disclosure. Note that the estimated reaction mechanism of the production method of the present disclosure will be described by use of examples of production of a heterodimeric protein, but, as described below, the production method of the present disclosure can also be applied to production of a product other than a heterodimeric protein. Accordingly, with the production method of the present disclosure, it is possible to produce a heteromultimeric protein.

### Embodiment 1

A production method of this embodiment will be described by use of examples of production of a heterodimeric protein using two proteins.

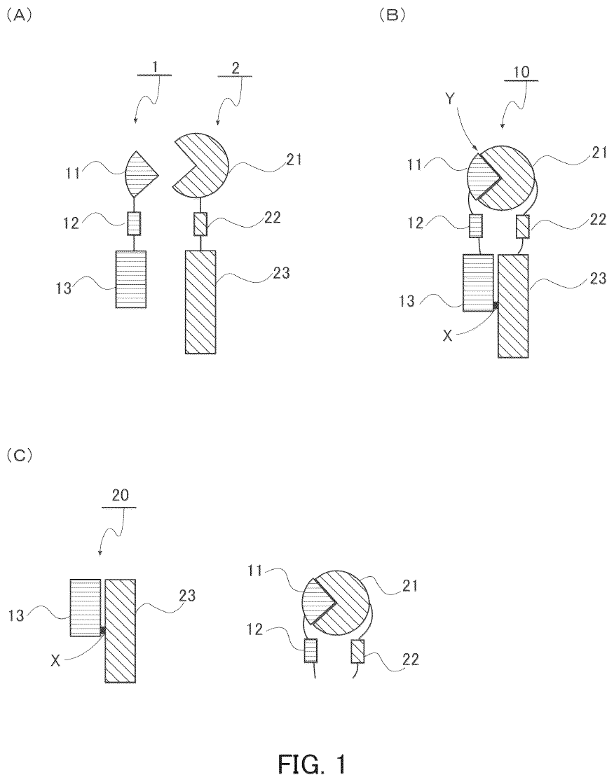

The following describes an example in which a heterodimeric protein 20 is produced using a first protein 1 and a second protein 2. As shown in FIG. 1(A), the first protein 1 includes a first binding tag 11, a first cleavable domain 12, and a first domain 13 in this order from the N terminus to the C terminus. The second protein 2 includes a first binding partner 21 capable of binding to the first binding tag 11, a second cleavable domain 22, and a second domain 23 in this order from the N terminus to the C terminus.

First, as shown in FIG. 1(B), the first protein 1 and the second protein 2 are brought into contact with each other. Thereby, a binding is established between the first domain 13 and the second domain 23 as indicated by X. Also, as indicated by an arrow Y, a binding between the first binding tag 11 and the first binding partner 21 is established. Thereby, the first protein 1 and the second protein 2 form a first complex 10. Next, as shown in FIG. 1(C), the first cleavable domain 12 and the second cleavable domain 22 of the first complex 10 are cleaved. Thereby, the first binding tag 11 and the first binding partner 21, which are located on the N terminus side relative to the first cleavable domain 12 and the second cleavable domain 22, respectively, separate from the first complex 10. As a result, as shown in FIG. 1(C), it is possible to produce the heterodimeric protein 20 that includes the first domain 13 and the second domain 23. Therefore, it is assumed that, with the production method of the present disclosure, a desired heterodimeric protein can be produced by locating monomeric proteins included in the desired heterodimer as the first domain 13 and the second domain 23.

In the production method of this embodiment, the first complex is formed by the first binding tag and the first binding partner binding together and the first domain and the second domain binding together. The first protein and the second protein can be prepared using, for example, a genetic engineering technique as described below in the description of a method for producing a protein according to the present disclosure. Accordingly, the production method of the present disclosure may optionally include a first expression step of allowing a host cell to express the first protein and the second protein prior to the complex formation step. The descriptions below about the protein, a nucleic acid, an expression vector, a transformant, and a method for producing a protein according to the present disclosure can be applied to an expression method in the expression step.

In the complex formation step, the first protein and the second protein are reacted. Thereby, as shown in FIG. 1(B), the first complex of the first protein and the second protein is formed in the complex formation step by the first binding tag and the first binding partner binding together and the first domain and the second domain binding together.

In the complex formation step, the first protein and the second protein may form a complex through, for example, (1) a binding between the first binding tag and the first binding partner, (2) a binding between the first domain and the second domain, or bindings of both (1) and (2), but it is preferable that the complex is formed through the bindings of both (1) and (2) because the dimer formation ability can be improved.

The binding between the first domain and the second domain may be a direct binding or an indirect binding (association), or may be formed through both the direct binding and the indirect binding between the first domain and the second domain, but the direct binding is preferable. The direct binding is a covalent bond, and specific examples thereof include an amide bond (e.g., a peptide bond or an isopeptide bond) between amino acids, a disulfide bond between cysteines, and the like. The indirect binding is a noncovalent bond, and specific examples thereof include a hydrogen bond, a hydrophobic bond, and the like.

The first domain and the second domain can have amino acid sequences capable of forming a dimer in a condition-dependent manner or in a condition-independent manner when proteins that include the domains coexist. In a specific example, the amino acid sequences of subunits in a protein dimer or the amino acid sequences of dimer formation motif sequences thereof can be used for the first domain and the second domain. The amino acid sequences of subunits in a protein multimer or the amino acid sequences of dimer formation motif sequences thereof may be used for the first domain and the second domain. The protein dimer may be a homodimer or a heterodimer. Examples of the protein dimer include dimers composed of a heavy chain and a light chain of immunoglobulins (antibodies) such as IgA, IgD, IgE, IgG, and IgM; proteins with a leucine zipper such as AP-1 (c-fos and c-jun) and myc family proteins (e.g., myc, max, mdx1, and the like); G protein-coupled receptors; kinesins; receptor-type tyrosine kinases such as the ErbB receptor family (e.g., an epidermal growth factor receptor (EGFR)), a platelet-derived growth factor receptor (PDGFR), a neurotrophin (neurotrophic factor) receptor, an insulin receptor, an insulin-like growth factor receptor, a vascular endothelial growth factor receptor (VEGFR), and a stem cell factor receptor; Toll-like receptors such as TLR1 to TLR11; and the like.

When the dimer formation motif sequences of an antibody are used as the first domain and the second domain, the first domain and the second domain include, for example, a light chain and a heavy chain of an antibody that binds to a first target antigen, respectively.

The antibody is, for example, IgA, IgD, IgE, IgG, or IgM, among which IgG is preferable. The IgG is, for example, IgG1, IgG2, IgG2a, IgG2b, IgG3, or IgG4. The antibody is, for example, an animal-derived antibody, and specific examples thereof include a human antibody, a mouse antibody, an avian antibody, a rat antibody, a rabbit antibody, and the like. The first domain and the second domain are preferably human-derived antibodies and more preferably human-derived IgGs.

Regarding the amino acid sequences of the human IgG1, IgG2, IgG3, and IgG4, for example, the amino acid sequences registered as UniProt Accession Nos. P01857, P01859, P01860, and P01861 can be referred to, respectively.

When the dimer formation motif sequences of an antibody are used as the first domain and the second domain, the antibody may be an antibody with an altered constant region. In this case, the first domain and the second domain may include an amino acid sequence of a light chain or heavy chain with the altered constant region. Examples of the constant region-altered antibody include Fcab (Fc antigen binding, References 1 and 5) to which an ability to bind to a target molecule is imparted by altering the amino acid sequence of the constant region, an IgG Hexamer (References 2 to 4) to which an ability to form a hexamer is imparted by altering the amino acid sequence of the constant region of an IgG antibody, DAF (Dual Action Fab, Reference 5), Charge pair (Amgen Inc., Reference 5), SEEDbody (Reference 5), Knobs-in-holes (Reference 5), DVI-IgG (Reference 5), and the like.
Reference 1: G. Wozniak-Knopp et al., “Introducing antigen-binding sites in structural loops of immunoglobulin constant domains: Fc fragments with engineered HER2/neu-binding sites and antibody properties”, Protein Engineering. Design and Selection, Volume 23, Issue 4, April 2010, Pages 289-297
Reference 2: Sopp, J. M. et al., “On-target IgG hexamerisation driven by a C-terminal IgM tail-piece fusion variant confers augmented complement activation.”, Commun. Biol., 4, 1031 (2021).
Reference 3: de Jong R N et al., “A Novel Platform for the Potentiation of Therapeutic Antibodies Based on Antigen-Dependent Formation of IgG Hexamers at the Cell Surface.”, PLoS. Biol. (2016) 14(1): e1002344.
Reference 4: Christoph A. Diebolder et al., “Complement Is Activated by IgG Hexamers Assembled at the Cell Surface”, Science, 343 (6176), pages 1260-1263
Reference 5: Christoph Spiess et al., “Alternative molecular formats and therapeutic applications for bispecific antibodies”, Molecular Immunology, Volume 67, Issue 2, Part A, 2015, Pages 95-106

The first binding tag and the first binding partner are such molecules that the first binding tag and the first binding partner bind together in a condition-dependent manner or in a condition-independent manner when a protein that includes the first binding tag and a protein that includes the first binding partner coexist. The binding between the first binding tag and the first binding partner may be a direct binding or an indirect binding.

When the binding between the first binding tag and the first binding partner is a direct binding, for example, a peptide tag and a peptide that can spontaneously form a covalent bond or a peptide tag and a peptide that can form a covalent bond due to modification activity of another molecule can be used as the first binding tag and the first binding partner.

When the peptide tag and the peptide that can spontaneously form a covalent bond are used, examples of the first binding tag and the first binding partner include: a Streptococcus pyogenes surface protein (SpyCatcher, SEQ ID NO: 1) and a peptide tag (SpyTag, SEQ ID NO: 2) capable of binding to the SpyCatcher, or altered products thereof; a Streptococcus pneumoniae protein (SnoopCatcher, SEQ ID NO: 3) and a peptide tag (SnoopTag, SEQ ID NO: 4) capable of binding to the SnoopCatcher, or altered products thereof; an altered Clostridium perfringens protein Cpe0147₄₃₉₋₅₆₃ (SEQ ID NO: 5) and a peptide tag Cpe0147₅₆₅₋₅₈₇ (SEQ ID NO: 6) capable of binding to the Cpe0147₄₃₉₋₅₆₃, or altered products thereof; and the like. Examples of the altered products of the SpyCatcher and the SpyTag include SpyCatcher2 and SpyTag2 (Reference 6), SpyCatcher3 and SpyTag3 (Reference 7), SnoopCatcher and SnoopTag (Reference 8), and the like. These peptide tags and the peptides that can spontaneously form a covalent bond bind together via, for example, an isopeptide bond.
Reference 6: Anthony H. Keeble et al., “Evolving Accelerated Amidation by SpyTag/SpyCatcher to Analyze Membrane Dynamics”, Angew. Chem. Int. Ed., 2017, 56, pages 16521-16525
Reference 7: Anthony H. Keeble et al., “Approaching infinite affinity through engineering of Peptide-protein interaction”, PNAS, 2019, vol. 116, No. 52, pages 26523-26533
Reference 8: Veggiani G, Nakamura T, Brenner M D, Gayet R V, Yan J, Robinson C V, Howarth M. Programmable polyproteams built using twin peptide superglues. Proc Natl Acad Sci USA. 2016 Feb. 2; 113(5): 1202-7. doi: 10.1073/pnas.1519214113.
   Amino Acid Sequence of Streptococcus pyogenes Surface Protein (SpyCatcher) (SEQ ID NO: 1)

DSATHIKFSKRDEDGKELAGATMELRDSSGKTISTWISDGQ
VKDFYLYPGKYTFVETAAPDGYEVATAITFTVNEQGQVTVN

### Amino Acid Sequence of SpyTag (SEQ ID NO: 2)

AHIVMVDAYKPTK

Amino Acid Sequence of Streptococcus pneumoniae Protein (SnoopCatcher) (SEQ ID NO: 3)

KPLRGAVFSLQKQHPDYPDIYGAIDQNGTYQNVRTGEDGKLTFKN
LSDGKYRLFENSEPAGYKPVQNKPIVAFQIVNGEVRDVTSIVPQD
IPATYEFTNDKHYITNEPIPPK

### Amino Acid Sequence of SnoopTag (SEQ ID NO: 4)

KLGDIEFIKVNK

Amino Acid Sequence of Altered Clostridium perfringens Protein Cpe0147₄₃₉₋₅₆₃ (SEQ ID NO: 5)

MTLKTTVAADGVNGSSEKEALVSFENSKDGVDVKDTIDYKDLVAN
EKYNLTGKLMHVKDDGSLEEVATKTTEVTAVENGSGQWELDFGNQ
KLQVGEKYVVFENAESVENLIDTDNNYELDTK

### Amino Acid Sequence of Peptide Tag Cpe0147₅₆₅₋₅₈₇ (SEQ ID NO: 6)

QVVKHEDKNDKAQTLIVEKPLE

When the peptide tag and the peptide that can form a covalent bond due to modification activity of the other molecule are used, examples of the first binding tag and the first binding partner include a K tag and a Q tag, and the like. A covalent bond can be formed between the K tag and the Q tag by, for example, forming a cross-link between the lysine residue at the N terminus of the K tag and the glutamic acid at the N terminus of the Q tag using bacterial transglutaminase.

When the binding between the first binding tag and the first binding partner is an indirect binding, for example, an affinity tag and a molecule that binds to the affinity tag can be used as the first binding tag and the first binding partner. The first binding tag and the first binding partner are, for example, peptides, polypeptides, or proteins. Examples of the binding tag include a His-tag (His×6), a His-Strep-tag, a strep-tag, an avidin tag, a flag (trademark)-tag, an HA (hemagglutinin)-tag, a T7-tag, a V5-peptide-tag, a GST (glutathione-S-transferase)-tag, a CBP (calmodulin-binding peptide)-tag, an MBP (maltose-binding protein)-tag, a Myc-tag, and the like. When the first binding partner is a molecule that specifically binds to a target molecule such as an antibody or an antigen-binding fragment thereof, or a derivative thereof, the binding tag may be a peptide having any amino acid sequence to which the molecule that specifically binds to the target molecule can bind.

The first binding partner can be selected as appropriate in accordance with the type of the first binding tag. Specific examples of the first binding partner include an antibody that recognizes the first binding tag or an antigen-binding fragment thereof, or a derivative thereof; a nucleic acid molecule such as an aptamer; glutathione, calmodulin; a sugar chain such as mannose; a metal such as nickel, cobalt, or zinc or an ion thereof; and the like.

The combination of the first binding tag and the first binding partner need only be a combination that enables binding between the first binding tag and the first binding partner. In a specific example in which the first binding tag includes a His-tag, the first binding partner is, for example, nickel. When the first binding tag includes a strep-tag or an avidin tag, the first binding partner includes, for example, biotin. When the first binding tag includes an epitope tag such as a flag (trademark)-tag, an HA-tag, a T7-tag, a V5-peptide-tag, and/or a Myc-tag, examples of the first binding partner include an antibody against the epitope tag or an antigen-binding fragment thereof, or a derivative thereof, or the like. When the first binding tag includes a GST-tag, the first binding partner is, for example, glutathione. When the first binding tag includes a CBP-tag, the first binding partner is, for example, calmodulin. When the first binding tag includes an MBP-tag, the first binding partner is, for example, mannose.

Functional equivalents may be used as the first binding tag and the first binding partner as long as an ability to establish a binding between the first binding tag and the first binding partner is maintained. When the first binding tag or the first binding partner is a peptide, a polypeptide, or a protein, the functional equivalent may be, for example, a polypeptide that has an amino acid sequence having 70% or more, 80% or more, 85% or more, 90% or more, 95% or more, 96% or more, 97% or more, 98% or more, or 99% or more identity to the reference amino acid sequence of the first binding tag or the first binding partner and that has an ability to bind to the corresponding first binding tag or first binding partner. The functional equivalents may be, for example, polypeptides that have amino acid sequences consisting of the reference amino acid sequences of the first binding tag and the first binding partner with deletion, substitution, insertion, and/or addition of one or several amino acids and that have an ability to bind to the corresponding binding tag or binding partner. The “one or several” amino acids above refer to, for example, 1 to 44 amino acids, 1 to 33 amino acids, 1 to 22 amino acids, 1 to 11 amino acids, 1 to 8 amino acids, 1 to 6 amino acids, 1 to 4 amino acids, 1 to 3 amino acids, 1 or 2 amino acids, or 1 amino acid. The substitution is preferably, for example, conservative substitution.

The first protein may have one first binding tag or a plurality of first binding tags. In the latter case, one type of first binding tag or a plurality of types of first binding tags may be used. It is preferable that, in the first protein, the first binding tag is located on the N terminus side of the first protein.

The second protein may have one first binding partner or a plurality of first binding partners. In the latter case, one type of first binding partner or a plurality of types of first binding partners may be used. It is preferable that, in the second protein, the first binding partner is located on the N terminus side of the second protein. In the proteins, the number of the first binding tags and the number of the first binding partners may be the same or different, but are preferably the same.

In the first protein and the second protein, the first binding tag and the first binding partner are interchangeable, and those included in the combination in the description above may be interchanged and used.

The first cleavable domain and the second cleavable domain are domains having such an amino acid sequence that causes cleavage in the domain in a condition-dependent manner or in a condition-independent manner. The cleavage is caused in a generation step, which will be described below, after the formation of the first complex. Accordingly, it is preferable that the first cleavable domain and the second cleavable domain are cleavable domains that are cleaved in a condition-dependent manner.

The first cleavable domain and the second cleavable domain may be the same cleavable domain or different cleavable domains. The numbers of the first cleavable domains and the second cleavable domains each may be one or more. In the latter case, one type of cleavable domain or a plurality of types of cleavable domains may be used. Using the same cleavable domain as the first cleavable domain and the second cleavable domain makes it possible to, for example, cleave both of the cleavable domains in a single reaction in the generation step, which will be described below, and thus a heterodimeric protein can be efficiently produced.

It is preferable that the first cleavable domain and/or the second cleavable domain includes a cleavable sequence of a self-cleaving peptide and/or a sequence capable of being cleaved by a protease or peptidase. Examples of the self-cleaving peptide include a 2A self-cleaving peptide and intein, or altered products thereof. Examples of the sequence capable of being cleaved by a protease or peptidase include a thrombin cleavage sequence, a Factor Xa recognition sequence, a GST fusion protein cleavage enzyme (PreScission (trademark) Protease) recognition sequence, a furin sensitive sequence, a carboxypeptidase sensitive sequence, and the like. It is preferable that the first cleavable domain and/or the second cleavable domain includes a sequence capable of being cleaved by a protease or peptidase because nonspecific cleavage can be suppressed.

In the first protein, the order of the first binding tag and the first cleavable domain can be determined, for example, in accordance with their positions relative to the first domain. The position at which the first cleavable domain is located is closer to the first domain than the position at which the first binding tag is located is. This allows the first binding tag to separate from the first domain when the first cleavable domain is cleaved in the generation step, which will be described below. Accordingly, in the production method of this embodiment, the first binding tag, the first cleavable domain, and the first domain are located, for example, in this order from the N terminus to the C terminus in the first protein.

In the second protein, the order of the first binding partner and the second cleavable domain can be determined, for example, in accordance with their positions relative to the second domain. The position at which the second cleavable domain is located is closer to the second domain than the position at which the first binding partner is located is. This allows the first binding partner to separate from the second domain when the second cleavable domain is cleaved in the generation step, which will be described below. Accordingly, in the production method of this embodiment, the first binding partner, the second cleavable domain, and the second domain are located, for example, in this order from the N terminus to the C terminus in the second protein.

In the first protein, the first binding tag, the first cleavable domain, and the first domain are directly or indirectly linked to one another. In the second protein, the first binding partner, the second cleavable domain, and the second domain are directly or indirectly linked to one another.

The “direct binding” means that an N-terminal or C-terminal amino acid of one polypeptide or domain forms a peptide bond together with a C-terminal or N-terminal amino acid of another polypeptide or domain and binds thereto. On the other hand, the “indirect binding” means that an N-terminal or C-terminal amino acid of one polypeptide or domain binds to a C-terminal or N-terminal amino acid of another polypeptide or domain via a linker peptide (peptide linker), that is, an N-terminal or C-terminal amino acid of one polypeptide or domain forms a peptide bond together with a C-terminal or N-terminal amino acid of the linker peptide and binds thereto while an amino acid at the other terminus of the linker peptide binds to an N-terminal or C-terminal amino acid of another polypeptide or domain. The linker peptide is composed of, for example, 5 to 15 amino acids. A known linker peptide can be used as the linker peptide, and specific examples thereof include GS linkers (GS, GGS, and GGGGS (SEQ ID NO: 7)), linker peptides formed by repeatedly linking GS linkers together ([GS]ₗ, [GGS]ₘ, or [GGGGS]ₙ (l, m, n are integers greater than or equal to 2)), GGGSGG (SEQ ID NO: 8), and the like.

The first protein may include, for example, other polypeptides such as a solubilization domain and a signal peptide on the N terminus side of the first binding tag. The second protein may include, for example, other polypeptides such as a solubilization domain and a signal peptide on the N terminus side of the first binding partner. The solubilization domain is preferably a polypeptide that, when fused to the polypeptide or protein, increases the expression level of the target protein such as the first protein or second protein expressed in a transformant, which will be described later, by at least 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 100% or more compared with expression of the polypeptide or protein without the solubilization domain. The solubilization domain is, for example, a protein or a partial polypeptide thereof. Specific examples of the solubilization domain include GST, MBP, thioredoxin, antibodies, altered antibodies such as a single-chain antibody, and the like.

When the first protein includes the solubilization domain, the first protein may include one solubilization domain or a plurality of solubilization domains. In the latter case, one type of solubilization domain or a plurality of types of solubilization domains may be used.

When the second protein includes the solubilization domain, the second protein may include one solubilization domain or a plurality of solubilization domains. In the latter case, one type of solubilization domain or a plurality of types of solubilization domains may be used.

The first protein and the second protein may include, for example, a purification tag for use to purify the first protein, the second protein, or the heterodimeric protein. For example, the affinity tag described above can be used as the purification tag. The purification tag can be added to, for example, at least one of the N terminus (side) and the C terminus (side) of the first domain and at least one of the N terminus (side) and the C terminus (side) of the second domain.

The reaction conditions in the complex formation step need only be conditions under which the first protein and the second protein can form a dimer, and can be determined as appropriate in consideration of the reaction conditions (binding conditions) for the binding tag and the binding partner and/or the reaction conditions (binding conditions) for the first domain and the second domain. A specific example of the reaction temperature in the complex formation step is 4° C. to 50° C. or 10° C. to 45° C. The reaction time in the complex formation step is, for example, 0.1 to 24 hours or 0.5 to 12 hours. The reaction pH in the complex formation step is, for example, pH 4 to 10 or pH 5 to 9.

When the binding tag and the binding partner directly bind together, the reaction conditions in the complex formation step can be set to reaction conditions under which a binding between the binding tag and the binding partner is sufficiently established. In a specific example in which the SpyTag and the SpyCatcher are used as the binding tag and the binding partner, the complex formation step can be conducted, for example, at pH 5 to 8 at 4° C. to 37° C. in the presence of a buffer solution. When the SnoopCatcher and the SnoopTag are used as the binding tag and the binding partner, the complex formation step can be conducted, for example, at pH 5 to 8 at 4° C. to 37° C. in the presence of a buffer solution. When a binding between the binding tag and the binding partner is generated through an enzyme reaction, the reaction conditions in the complex formation step can be determined, for example, based on the conditions under which an enzyme used in the enzyme reaction exhibits its activity.

When the first domain and the second domain directly bind together, the reaction conditions in the complex formation step can be set to reaction conditions under which a binding between the first domain and the second domain is sufficiently established. In a specific example in which the first domain and the second domain bind together via a disulfide bond, the reaction conditions in the complex formation step can be set to those under which the disulfide bond is not reduced. In another specific example in which the first protein and the second protein bind together via an isopeptide bond, the reaction conditions in the complex formation step can be set to those under which the isopeptide bond is not hydrolyzed.

When the heterodimeric protein is a part of a multispecific antibody, the reaction conditions in the complex formation step may be conditions under which the first protein and the second protein can further form disulfide bonds between the heavy chain and the light chain.

The production method of this embodiment may include a first purification step of purifying the first complex after the complex formation step. For example, a common protein purification method such as chromatography can be utilized as the purification method in the purification step.

Next, in the generation step, the first cleavable domain and the second cleavable domain of the first complex are cleaved. Thereby, as shown in FIG. 1(C), in the generation step, the first binding tag and the first binding partner binding together separate, and a heterodimeric protein composed of the first domain and the second domain is generated.

In the generation step, the cleavage reaction conditions (e.g., the reaction temperature, the reaction time, the reaction pH, and the like) can be set to, for example, those under which the first cleavable domain and the second cleavable domain undergo the cleavage reaction. In a specific example in which the first cleavable domain and the second cleavable domain are the thrombin cleavage sequences, the cleavage reaction temperature is, for example, 0° C. to 40° C., 4° C. to 37° C., or 4° C. to 30° C. The cleavage reaction time is, for example, 1 minute to 48 hours, 30 minutes to 48 hours, or 1 to 48 hours. The cleavage reaction pH is, for example, pH 5 to 10, pH 6 to 9, or pH 6.5 to 9.

When the first cleavable domain and/or the second cleavable domain is a domain capable of being cleaved by a protease or peptidase in the generation step, the generation step may be conducted in the presence of the protease or peptidase. In this case, the generation step can be conducted under the reaction conditions under which the protease or the peptidase exhibits cleavage activity.

The production method of the present disclosure may include a second purification step of purifying the heterodimeric protein after the generation step. For example, a common protein purification method such as chromatography can be utilized as the purification method in the purification step.

Thereby, with the production method of this embodiment, it is possible to produce a heterodimeric protein using two proteins.

Although the production method of this embodiment has been described by use of examples of production of a heterodimeric protein using two isolated proteins, the present disclosure is not limited thereto, and the first complex may be produced using two proteins in a host cell, which will be described below. In this case, in the production method of this embodiment, the first complex can be formed in the host cell by allowing the host cell to express the first protein and the second protein. Moreover, in the production method of this embodiment, a heterodimeric protein can be produced by, for example, purifying the first complex from the host cell and conducting the generation step.

In the production method of this embodiment, both of the two proteins are monomeric proteins, but the present disclosure is not limited thereto, and one or both of them may be proteins composed of two or more components, that is, the multimeric proteins. In a specific example in which a heterotrimeric protein is produced as the heteromultimeric protein, the trimeric protein can be produced by using one protein to be included in the trimeric protein as the first domain and a dimer composed of the other two proteins to be included in the trimeric protein as the second domain. Therefore, with the production method of the present disclosure, it is possible to produce a desired heteromultimeric protein.

### Embodiment 2

Next, a production method of this embodiment will be described by use of examples of production of a heterotetrameric protein using four proteins. In the production method of this embodiment, a third protein and a fourth protein as well as the first protein 1 and the second protein 2 in the production method of Embodiment 1 are included as proteins for producing a heteromultimeric protein, and a heterotetrameric protein is produced using these proteins. Accordingly, the descriptions of the various configurations and steps in the production method of Embodiment 1 can be applied to the descriptions of various configurations and steps in the production method of Embodiment 2 unless otherwise stated.

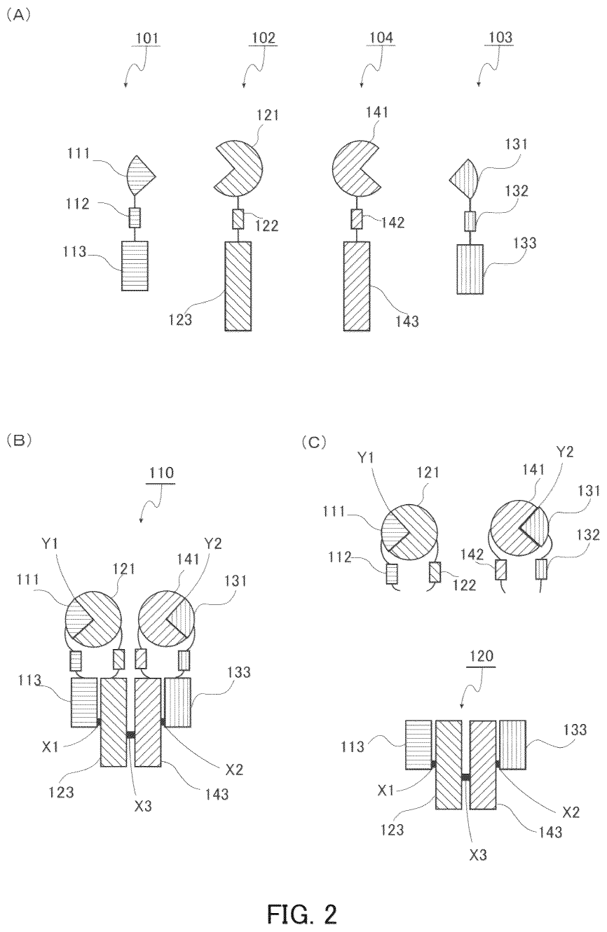

In the production method of this embodiment, a third protein 103 and a fourth protein 104 are included in addition to the two proteins used in the production method of Embodiment 1. The production method of this embodiment will be described by use of examples of production of a heterotetrameric protein 120 using a first protein 101, a second protein 102, the third protein 103, and the fourth protein 104. As shown in FIG. 2(A), the first protein 101 includes a first binding tag 111, a first cleavable domain 112, and a first domain 113 in this order from the N terminus to the C terminus. The second protein 102 includes a first binding partner 121 capable of binding to the first binding tag 111, a second cleavable domain 122, and a second domain 123 in this order from the N terminus to the C terminus. The third protein 103 includes a second binding tag 131, a third cleavable domain 132, and a third domain 133 in this order from the N terminus to the C terminus. The fourth protein 104 includes a second binding partner 141 capable of binding to the second binding tag 131, a fourth cleavable domain 142, and a fourth domain 143 in this order from the N terminus to the C terminus.

First, as shown in FIG. 2(B), in the complex formation step, the first protein 101, the second protein 102, the third protein 103, and the fourth protein 104 are brought into contact with one another. Thereby, as indicated by X1, a binding is established between the first domain 113 and the second domain 123. Also, as indicated by X2, a binding is established between the third domain 133 and the fourth domain 143. Furthermore, as indicated by X3, a binding is established between the second domain 123 and the fourth domain 143. Then, a binding between the first binding tag 111 and the first binding partner 121 is established as indicated by Y1, and a binding between the second binding tag 131 and the second binding partner 141 is established as indicated by Y2. Thereby, the first protein 101, the second protein 102, the third protein 103, and the fourth protein 104 form a second complex 110. Next, as shown in FIG. 2(C), the first cleavable domain 112, the second cleavable domain 122, the third cleavable domain 132, and the fourth cleavable domain 142 of the second complex 110 are cleaved. Thereby, the first binding tag 111, the first binding partner 121, the second binding tag 131, and the second binding partner 141, which are located on the N terminus side relative to the first cleavable domain 112, the second cleavable domain 122, the third cleavable domain 132, and the fourth cleavable domain 142, respectively, separate from the second complex 110. As a result, as shown in FIG. 2(C), it is possible to produce the heterotetrameric protein 120 that includes the first domain 113, the second domain 123, the third domain 133, and the fourth domain 143. Therefore, it is assumed that, with the production method of this embodiment, it is possible to produce a desired heterotetrameric protein by locating monomeric proteins included in the desired heterotetramer at the first domain 113, the second domain 123, the third domain 133, and the fourth domain 143.

In the production method of this embodiment, the second complex is formed by the first binding tag and the first binding partner binding together, the second binding tag and the second binding partner binding together, the first domain and the second domain binding together, the second domain and the fourth domain binding together, and the third domain and the fourth domain binding together. The first protein, the second protein, the third protein, and the fourth protein can be prepared using, for example, a genetic engineering technique as described below in the description of a method for producing a protein according to the present disclosure. Accordingly, the production method of the present disclosure may optionally include a first expression step of allowing a host cell to express the first protein, the second protein, the third protein, and the fourth protein prior to the complex formation step. The descriptions below about the protein, a nucleic acid, an expression vector, a transformant, and a method for producing a protein according to the present disclosure can be applied to an expression method in the expression step.

In the complex formation step, the first protein, the second protein, the third protein, and the fourth protein are reacted. Thereby, as shown in FIG. 2(B), in the complex formation step, the first protein, the second protein, the third protein, and the fourth protein form the second complex by the first binding tag and the first binding partner binding together, the second binding tag and the second binding partner binding together, the first domain and the second domain binding together, the second domain and the fourth domain binding together, and the third domain and the fourth domain binding together.

In the complex formation step, the first to fourth proteins may form a complex through, for example, (1) a binding between the binding tag and the binding partner, namely a binding between the first binding tag and the first binding partner and/or a binding between the second binding tag and the second binding partner, (2) a binding between the domains, namely a binding between the first domain and the second domain, a binding between the second domain and the fourth domain, and/or a binding between the third domain and the fourth domain, or bindings of both (1) and (2), but it is preferable that the complex is formed through the bindings of both (1) and (2) because the dimer formation ability can be improved.

The binding between the first domain and the second domain, the binding between the second domain and the fourth domain, and/or the binding between the third domain and the fourth domain may be a direct binding or an indirect binding (association), or may be formed through both the direct binding and the indirect binding between the first domain and the second domain, but the direct binding is preferable. The direct binding is a covalent bond, and specific examples thereof include an amide bond (e.g., a peptide bond or an isopeptide bond) between amino acids, a disulfide bond between cysteines, and the like. The indirect binding is a noncovalent bond, and specific examples thereof include a hydrogen bond, a hydrophobic bond, and the like.

The first to fourth domains can have amino acid sequences capable of forming a tetramer in a condition-dependent manner or in a condition-independent manner when proteins that include the domains coexist. In a specific example, the amino acid sequences of subunits in a protein tetramer or the amino acid sequences of dimer formation motif sequences thereof can be used for the first to fourth domains. The amino acid sequences of subunits in a protein multimer or the amino acid sequences of tetramer formation motif sequences thereof may be used for the first to fourth domains. The protein tetramer may be a heterotetramer in which some of the subunits are the same, or a heterotetramer in which all the subunits are different. Examples of the protein tetramer include immunoglobulins (antibodies) such as IgA, IgD, IgE, IgG, and IgM; small bispecific antibodies (i.e., diabodies); and the like. When the antibody subunits are used as the first to fourth domains, the antibody may be an antibody with an altered constant region.

When the tetramer formation motif sequences of antibodies are used as the first to fourth domains, the first domain is a light chain of an antibody that binds to a first target, the second domain is a heavy chain of the antibody that binds to the first target, the third domain is a light chain of an antibody that binds to a second target, and the fourth domain is a heavy chain of the antibody that binds to the second target in an example. The antibody that binds to the first target and the antibody that binds to the second target may recognize the same antigen or different antigens, but preferably recognize different antigens. The antibody that binds to the first target and the antibody that binds to the second target may recognize the same epitope or different epitopes, but preferably recognize different epitopes. With the production method of the present disclosure, it is possible to favorably produce, for example, a bispecific antibody by using antibodies that recognize different antigens or epitopes as the antibody that binds to the first target and the antibody that binds to the second target. When the tetramer formation motif sequences of antibodies are used as the first to fourth domains, the first domain and the third domain are light chains of antibodies, and the second domain and the fourth domain are heavy chains of antibodies, it is preferable that heavy chains of antibodies in which a constant region, which causes specific association, is altered are used as the second domain and the fourth domain. Examples of the antibodies in which a constant region, which causes specific association, is altered include Charge pair, Knobs-in-holes, and the like.

The first binding tag and the first binding partner are such molecules that the first binding tag and the first binding partner bind together in a condition-dependent manner or in a condition-independent manner when a protein that includes the first binding tag and a protein that includes the first binding partner coexist. The binding between the first binding tag and the first binding partner may be a direct binding or an indirect binding. The second binding tag and the second binding partner are such molecules that the second binding tag and the second binding partner bind together in a condition-dependent manner or in a condition-independent manner when a protein that includes the second binding tag and a protein that includes the second binding partner coexist. The binding between the second binding tag and the second binding partner may be a direct binding or an indirect binding. In the production method of this embodiment, the binding between the first binding tag and the first binding partner and the binding between the second binding tag and the second binding partner are preferably direct bindings. Thereby, it is possible to suppress, for example, nonspecific binding between a binding tag and a binding partner in the production method of this embodiment, thus making it possible to efficiently produce a desired heterotetramer.

The examples of the first binding tag and the first binding partner shown in Embodiment 1 can be applied to specific examples of the second binding tag and the second binding partner.

The first binding tag and the second binding tag are configured to be capable of binding to, for example, the first binding partner and the second binding partner, respectively. Specifically, the first binding tag and the second binding tag are configured such that the binding between the first binding tag and the second binding partner is, for example, less specific than the binding between the first binding tag and the first binding partner, and the binding between the second binding tag and the first binding partner is less specific than the binding between the second binding tag and the second binding partner. Thereby, the first protein can specifically bind to the second protein, and the third protein can specifically bind to the fourth protein. In a specific example, it is preferable that the combination of the first binding tag and the first binding partner and the combination of the second binding tag and the second binding partner are different from each other.

The third protein may have one second binding tag or a plurality of second binding tags. In the latter case, one type of second binding tag or a plurality of types of second binding tags may be used. It is preferable that, in the third protein, the second binding tag is located on the N terminus side of the third protein.

The fourth protein may have one second binding partner or a plurality of second binding partners. In the latter case, one type of second binding partner or a plurality of types of second binding partners may be used. It is preferable that, in the fourth protein, the second binding partner is located on the N terminus side of the fourth protein. The number of the second binding tags in the third protein and the number of the second binding partners in the fourth protein may be the same or different, but are preferably the same.

In the third protein and the fourth protein, the second binding tag and the second binding partner are interchangeable, and those included in the combination in the description above may be interchanged and used.

The first to fourth cleavable domains are domains that are cleaved in a condition-dependent manner or in a condition-independent manner. The cleavage is caused in a generation step, which will be described below, after the formation of the second complex. Accordingly, it is preferable that the first to fourth cleavable domains are cleavable domains that are cleaved in a condition-dependent manner.

The descriptions of the first cleavable domain and the second cleavable domain in Embodiment 1 can be applied to specific examples of the third cleavable domain and the fourth cleavable domain. It is preferable that the first to fourth cleavable domains include a sequence capable of being cleaved by a protease or peptidase because nonspecific cleavage can be suppressed.

Some or all of the first to fourth cleavable domains may be the same cleavable domain or different cleavable domains. The numbers of the first to fourth cleavable domains may be one or more in the first to fourth proteins, respectively. In the latter case, one type of cleavable domain or a plurality of types of cleavable domains may be used. Using the same cleavable domain as the first to fourth cleavable domains makes it possible to, for example, cleave the first to fourth cleavable domains in a single reaction in the generation step, which will be described below, and thus a tetrameric protein can be efficiently produced.

In the third protein, the order of the second binding tag and the third cleavable domain can be determined, for example, in accordance with their positions relative to the third domain. The position at which the third cleavable domain is located is closer to the third domain than the position at which the second binding tag is located is. This allows the second binding tag to separate from the third domain when the third cleavable domain is cleaved in the generation step, which will be described below. Accordingly, in the production method of this embodiment, the second binding tag, the third cleavable domain, and the third domain are located, for example, in this order from the N terminus to the C terminus in the third protein.

In the fourth protein, the order of the second binding partner and the fourth cleavable domain can be determined, for example, in accordance with their positions relative to the fourth domain. The position at which the fourth cleavable domain is located is closer to the fourth domain than the position at which the second binding partner is located is. This allows the second binding partner to separate from the fourth domain when the fourth cleavable domain is cleaved in the generation step, which will be described below. Accordingly, in the production method of this embodiment, the second binding partner, the fourth cleavable domain, and the fourth domain are located, for example, in this order from the N terminus to the C terminus in the fourth protein.

In the third protein, the second binding tag, the third cleavable domain, and the third domain are directly or indirectly linked to one another. In the fourth protein, the second binding partner, the fourth cleavable domain, and the fourth domain are directly or indirectly linked to one another. The descriptions of the direct binding and the indirect binding in the descriptions of the first protein and the second protein in Embodiment 1 can be applied to the direct binding and the indirect binding above.

The third protein may include, for example, other polypeptides such as the solubilization domain and the signal peptide on the N terminus side of the second binding tag. The fourth protein may include, for example, other polypeptides such as the solubilization domain and the signal peptide on the N terminus side of the second binding partner.

When the third protein includes the solubilization domain, the third protein may include one solubilization domain or a plurality of solubilization domains. In the latter case, one type of solubilization domain or a plurality of types of solubilization domains may be used.

When the fourth protein includes the solubilization domain, the fourth protein may include one solubilization domain or a plurality of solubilization domains. In the latter case, one type of solubilization domain or a plurality of types of solubilization domains may be used.

The third protein and the fourth protein may include, for example, a purification tag for use to purify the third protein, the fourth protein, or the heterotetrameric protein. For example, the affinity tag described above can be used as the purification tag. The purification tag can be added to, for example, at least one of the N terminus (side) and the C terminus (side) of the third domain and at least one of the N terminus (side) and the C terminus (side) of the fourth domain.

The reaction conditions in the complex formation step need only be conditions under which the first to fourth proteins can form a tetramer, and can be determined as appropriate in consideration of the reaction conditions (binding conditions) for the binding tag and the binding partner and/or the reaction conditions (binding conditions) for the first to fourth domains.

When the third domain and the fourth domain directly bind together, the reaction conditions in the complex formation step can be set to reaction conditions under which a binding between the third domain and the fourth domain is sufficiently established. In a specific example in which the third domain and the fourth domain bind together via a disulfide bond, the reaction conditions in the complex formation step can be set to those under which the disulfide bond is not reduced. In another specific example in which the third protein and the fourth protein bind together via an isopeptide bond, the reaction conditions in the complex formation step can be set to those under which the isopeptide bond is not hydrolyzed. When the second domain and the fourth domain directly bind together, the reaction conditions in the complex formation step can be set to reaction conditions under which a binding between the second domain and the fourth domain is sufficiently established. In a specific example in which the second domain and the fourth domain bind together via a disulfide bond, the reaction conditions in the complex formation step can be set to those under which the disulfide bond is not reduced. In another specific example in which the second protein and the fourth protein bind together via an isopeptide bond, the reaction conditions in the complex formation step can be set to those under which the isopeptide bond is not hydrolyzed.

When the heterotetrameric protein is a multispecific antibody, the reaction conditions in the complex formation step may be conditions under which the first to fourth proteins can further form disulfide bonds between the heavy chain and the light chain and between the heavy chains.

The production method of the present disclosure may include a first purification step of purifying the second complex after the complex formation step. For example, a common protein purification method such as chromatography can be utilized as the purification method in the purification step.

Next, in the generation step, the first cleavable domain, the second cleavable domain, the third cleavable domain, and the fourth cleavable domain of the second complex are cleaved. Thereby, as shown in FIG. 2(C), in the generation step, the first binding tag and the first binding partner binding together and the second binding tag and the second binding partner binding together separate, and a heterotetramer composed of the first domain, the second domain, the third domain, and the fourth domain is generated.

In the generation step, the cleavage reaction conditions (e.g., the reaction temperature, the reaction time, the reaction pH, and the like) can be set to, for example, those under which the first to fourth cleavable domains undergo the cleavage reaction.

When the first cleavable domain, the second cleavable domain, the third cleavable domain, and/or the fourth cleavable domain is a domain capable of being cleaved by a protease or peptidase in the generation step, the generation step may be conducted in the presence of the protease or peptidase. In this case, the generation step can be conducted under the reaction conditions under which the protease or the peptidase exhibits cleavage activity.

The production method of the present disclosure may include a second purification step of purifying the heterotetrameric protein after the generation step. For example, a common protein purification method such as chromatography can be utilized as the purification method in the purification step.

Thereby, with the production method of this embodiment, it is possible to produce a heterotetrameric protein using four proteins.

Although the production method of this embodiment has been described by use of examples of production of a heterotetrameric protein from four isolated proteins, the present disclosure is not limited thereto, and the second complex may be produced using four proteins in a host cell, which will be described below. In this case, in the production method of this embodiment, the second complex can be formed in the host cell by allowing the host cell to express the first to fourth proteins. Moreover, in the production method of this embodiment, a heterotetrameric protein can be produced by, for example, purifying the second complex from the host cell and conducting the generation step.

In the production method of this embodiment, all of the four proteins are monomeric proteins, but the present disclosure is not limited thereto, and one or both of them may be proteins composed of two or more components, that is, the multimeric proteins.

In the production method of this embodiment, the second protein and the fourth protein associate and bind together via only the binding between the second domain and the fourth domain, but the second protein and the fourth protein may be configured to be capable of specifically binding together via another domain. In a specific example, the second protein may further include a fifth cleavable domain and a third binding tag in this order at the C terminus. Also, the fourth protein may further include a sixth cleavable domain and a third binding partner capable of binding to the third binding tag in this order at the C terminus. In this case, in the complex formation step, the third binding tag and the third binding partner also bind together. Then, in the generation step, for example, the fifth cleavable domain and the sixth cleavable domain of the second complex are also cleaved.

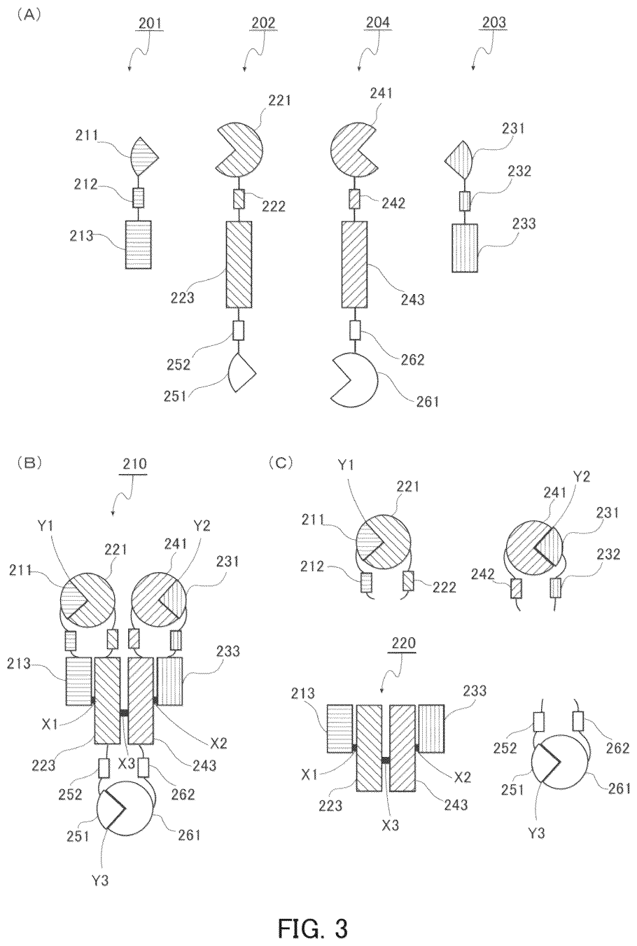

This will be described in more detail with reference to FIG. 3. First, as shown in FIG. 3(B), in the complex formation step, a first protein 201, a second protein 202, a third protein 203, and a fourth protein 204 are brought into contact with one another. Thereby, the binding tag and the binding partner of these proteins bind together. Specifically, as indicated by Y1, a first binding tag 211 and a first binding partner 221 bind together. Also, as indicated by Y2, a second binding tag 231 and a second binding partner 241 bind together. Furthermore, as indicated by Y3, a third binding tag 251 and a third binding partner 261 bind together. Meanwhile, a first domain 213, a second domain 223, a third domain 233, and a fourth domain 243 of these proteins also bind together. Specifically, as indicated by X1, the first domain 213 and the second domain 223 bind together. Also, as indicated by X2, the third domain 233 and the fourth domain 243 bind together. Furthermore, as indicated by X3, the second domain 223 and the fourth domain 243 bind together. As a result, in the complex formation step, the first protein 201, the second protein 202, the third protein 203, and the fourth protein 204 form a second complex 210.

Next, in the generation step, the first cleavable domain, the second cleavable domain, the third cleavable domain, the fourth cleavable domain, the fifth cleavable domain, and the sixth cleavable domain of the second complex are cleaved. Thereby, as shown in FIG. 3(C), in the generation step, the first binding tag 211 and the first binding partner 221 binding together, the second binding tag 231 and the second binding partner 241 binding together, and the third binding tag 251 and the third binding partner 261 binding together separate, and a heterotetramer 220 composed of the first domain 213, the second domain 223, the third domain 233, and the fourth domain 243 is generated.

The examples of the first binding tag and the first binding partner shown in Embodiment 1 can be applied to specific examples of the third binding tag and the third binding partner. The first binding tag, the second binding tag, and the third binding tag are configured to be capable of binding to, for example, the first binding partner, the second binding partner, and the third binding partner, respectively. Thereby, the first protein can bind to the second protein, the third protein can bind to the fourth protein, and the second protein can bind to the fourth protein. Accordingly, it is preferable that the combination of the first binding tag and the first binding partner, the combination of the second binding tag and the second binding partner, and the combination of the third binding tag and the binding partner are different from one another.

The examples of the first cleavable domain and the second cleavable domain shown in Embodiment 1 can be applied to specific examples of the fifth cleavable domain and the sixth cleavable domain. It is preferable that the first to sixth cleavable domains are the same cleavable domain.

### <Protein>

Another aspect of the present disclosure provides a protein that can be favorably used to produce the heteromultimeric protein. The protein of the present disclosure is a first protein, a second protein, a third protein, and/or a fourth protein, which will be described below.

The protein of the present disclosure (first protein) includes a first binding tag capable of binding to a first binding partner, a first cleavable domain, and a first domain in this order from the N terminus to the C terminus.

The protein of the present disclosure (second protein) includes the first binding partner capable of binding to the first binding tag, a second cleavable domain, and a second domain in this order from the N terminus to the C terminus.

The protein of the present disclosure (third protein) includes a second binding tag capable of binding to a second binding partner, a third cleavable domain, and a third domain in this order from the N terminus to the C terminus.

The protein of the present disclosure (fourth protein) includes the second binding partner capable of binding to the second binding tag, a fourth cleavable domain, and a fourth domain in this order from the N terminus to the C terminus.

The second protein and/or the fourth protein of the present disclosure may further include a cleavable domain and a binding tag or binding partner in this order on the C terminus side.

The first domain of the first protein and/or the third domain of the third protein according to the present disclosure is, for example, a polypeptide that includes an amino acid sequence of a light chain region of an immunoglobulin. The second domain of the second protein and/or the fourth domain of the fourth protein is, for example, a polypeptide that includes an amino acid sequence of a heavy chain region of an immunoglobulin.

### <Heteromultimeric Protein>

Another aspect of the present disclosure provides a heteromultimeric protein that can be favorably used to produce the heteromultimeric protein described previously. The protein (heteromultimeric protein) of the present disclosure includes two proteins,
wherein the two proteins include a first protein and a second protein,
the first protein includes a first binding tag, a first cleavable domain, and a first domain in this order from an N terminus to a C terminus,
the second protein includes a first binding partner capable of binding to the first binding tag, a second cleavable domain, and a second domain in this order from an N terminus to a C terminus,
the first protein and the second protein form a dimer via a binding between the first domain and the second domain, and
the first binding tag and the first binding partner bind together.

It is preferable that the heteromultimeric protein of the present disclosure further includes a third protein and a fourth protein,
wherein the third protein includes a second binding tag, a third cleavable domain, and a third domain in this order from an N terminus to a C terminus,
the fourth protein includes a second binding partner capable of binding to the second binding tag, a fourth cleavable domain, and a fourth domain in this order from an N terminus to a C terminus,
the second protein and the fourth protein form a dimer via a binding between the second domain and the fourth domain,
the third protein and the fourth protein form a dimer via a binding between the third domain and the fourth domain,
the first binding tag and the first binding partner bind together, and
the second binding tag and the second binding partner bind together.

When the heteromultimeric protein of the present disclosure is a bispecific antibody, the first domain of the first protein and the third domain of the third protein are each a polypeptide that includes an amino acid sequence of a light chain of an immunoglobulin. The second domain of the second protein and the fourth domain of the fourth protein are each a polypeptide that includes an amino acid sequence of a heavy chain of an immunoglobulin.

### <Nucleic Acid>

Another aspect of the present disclosure provides a nucleic acid that can be used to synthesize a heteromultimeric protein. The nucleic acid of the present disclosure encodes the protein and/or the heteromultimeric protein of the present disclosure.

The nucleic acid of the present disclosure need only encode any one or more of the proteins and the heteromultimeric protein of the present disclosure, and may encode two or more of them.

The nucleic acid of the present disclosure can be designed through replacement with corresponding codons based on the amino acid sequence of the heteromultimeric protein of the present disclosure. The base sequence of the nucleic acid of the present disclosure may be, for example, codon-optimized and is preferably codon-optimized to a host cell, which will be described below.

### <Expression Vector>

Another aspect of the present disclosure provides an expression vector that can be used to synthesize a protein and/or a heteromultimeric protein. The expression vector of the present disclosure includes the nucleic acid of the present disclosure. With the expression vector of the present disclosure, it is possible to favorably produce the proteins and/or the heteromultimeric protein of the present disclosure (also collectively referred to as “material proteins of the present disclosure” hereinafter) using a genetic engineering technique.

The expression vector of the present disclosure need only include a nucleic acid encoding any one or more of the proteins and the heteromultimeric protein of the present disclosure, and may include a nucleic acid encoding two or more of them.

The expression vector of the present disclosure is formed by, for example, inserting the nucleic acid of the present disclosure into an expression vector. The term “expression vector” means, for example, a nucleic acid molecule capable of transporting the inserted gene into a target such as a cell.

It is sufficient that the expression vector includes a polynucleotide of the nucleic acid of the present disclosure encoding the material protein of the present disclosure, for example, so as to be capable of expressing the material protein of the present disclosure encoded by the polynucleotide, and the configuration thereof is not particularly limited. For example, some or all of the polynucleotide encoding the material proteins of the present disclosure may be inserted into the same expression vector, or separate expression vectors. When some of the polynucleotide encoding the material proteins of the present disclosure are inserted into separate expression vectors, the expression vector of the present disclosure can be configured as an expression vector set containing expression vectors that each includes a nucleic acid encoding the material protein of the present disclosure.

The expression vector can be produced by, for example, inserting a polynucleotide encoding the protein of the present disclosure, that is, the nucleic acid of the present disclosure, into a skeleton vector (also referred to as a “basic vector” hereinafter). The type of the expression vector is not particularly limited and can be determined as appropriate in accordance with, for example, the type of the host.

Examples of the host cell (host) include non-human hosts such as microorganisms, animal cells, and insect cells, or cultured cells thereof, isolated human cells or cultured cells thereof, mammalian cells, and the like. Examples of the prokaryotes include bacteria belonging to the genus Escherichia such as Escherichia coli, and bacteria belonging to the genus Pseudomonas such as Pseudomonas putida. Examples of the eukaryotes include yeasts such as Saccharomyces cerevisiae, and the like. Examples of the animal cells include an HEK293 cell, an Expi293F cell, a COS cell, a CHO cell, and the like, and examples of the insect cells include Sf9, Sf21, and the like.

Examples of the expression vector (basic vector) include viral vectors and non-viral vectors. When the host is transformed using a heat shock method as the introduction method, the expression vector is, for example, a binary vector or the like. Examples of the expression vector include pETDuet-1, pQE-80L, pUCP26 Km, and the like. Examples of the expression vector for transformation of bacteria such as Escherichia coli include a pETDuet-1 vector (Novagen), pQE-80L (QIAGEN), pBR322, pB325, pAT153, pUC8, and the like. Examples of the expression vector for transformation of the yeasts include pYepSec1, pMFa, pYES2, and the like. Examples of the expression vector for transformation of the insect cells include pAc, pVL, and the like. Examples of the expression vector for transformation of the mammalian cells include pcDNA3.1, pcDNA3.4, pCAG, pCAGEN, pCDM8, pMT2PC, and the like.

It is preferable that the expression vector has a regulatory sequence that regulates, for example, the expression of the polynucleotide encoding the material protein of the present disclosure and the expression of the material protein of the present disclosure encoded by the polynucleotide for the material protein of the present disclosure. Examples of the regulatory sequence include a promoter, a terminator, an enhancer, a polyadenylation signal sequence, a replication origin sequence (ori), and the like. The position of the regulatory sequence in the expression vector is not particularly limited. It is sufficient that the regulatory sequence is placed in the expression vector so as to, for example, be capable of functionally regulating the expression of the polynucleotide encoding the material protein of the present disclosure and the expression of the material protein of the present disclosure encoded thereby, and the regulatory sequence can be placed based on a known method. The regulatory sequence may be one originally included in the basic vector, or the regulatory sequence may be further inserted into the basic vector, or a regulatory sequence included in the basic vector may be replaced with another regulatory sequence.

The expression vector may further have, for example, a sequence encoding a selection marker. Examples of the selection marker include a drug-resistance marker, a fluorescent protein marker, an enzyme marker, a cell-surface receptor marker, and the like.

DNA, the regulatory sequence, and/or the sequence encoding the selection marker may be inserted into the expression vector, for example, using a method in which a restriction enzyme and a ligase are used, or using a commercially available kit or the like.

### <Transformant>

Another aspect provides a transformant capable of producing the material protein of the present disclosure and a method for producing the transformant. The transformant of the present disclosure includes a nucleic acid encoding the material protein of the present disclosure. With the transformant of the present disclosure, it is possible to favorably produce the material protein of the present disclosure.

The method for producing a transformant according to the present disclosure includes a step of introducing the nucleic acid of the present disclosure into a host. With the method for producing a transformant according to the present disclosure, it is possible to produce the transformant above.

The descriptions of the nucleic acid encoding the material protein of the present disclosure can be applied to the nucleic acid encoding the material protein of the present disclosure in the transformant of the present disclosure. The expression vector of the present disclosure may be used as the nucleic acid of the present disclosure.

In the transformant of the present disclosure, the nucleic acid of the present disclosure is present as an exogenous molecule. Accordingly, the transformant of the present disclosure can be produced, for example, by introducing the nucleic acid of the present disclosure into the host.

The method for introducing the nucleic acid is not particularly limited and a known method can be used. The nucleic acid may be introduced, for example, using the expression vector. The introduction method can be determined as appropriate, for example, in accordance with the type of the host. Examples of the introduction method include an introduction method in which a gene gun such as a particle gun is used, a calcium phosphate method, a polyethylene glycol method, a lipofection method in which a liposome is used, an electroporation method, an ultrasonic nucleic acid introduction method, a DEAE-dextran method, a direct injection method in which a micro glass tube or the like is used, a hydrodynamic method, a cationic liposome method, a method in which an introduction auxiliary is used, a method in which an agrobacterium is used as a mediator, and the like. Examples of the liposome include Lipofectamine, a cationic liposome, and the like, and examples of the introduction auxiliary include atelocollagen, a nano particle, a polymer, and the like. When the host is a bacterium, for example, a method in which E. coli or Ps. putida is used as a mediator is particularly preferable. The polynucleotide encoding the protein of the present invention may be introduced into the host using, for example, the expression vector of the present disclosure.

### <Method for Producing Protein>

Another aspect of the present disclosure provides a method for producing a protein, and/or a protein that can be favorably used to produce a heteromultimeric protein, and/or a heteromultimeric protein. The method for producing a protein according to the present disclosure includes an expression step of expressing the nucleic acid of the present disclosure, the expression vector of the present disclosure, and/or the expression vector set of the present disclosure. With the method for producing a protein according to the present disclosure, it is possible to produce the material protein of the present disclosure.

The material protein of the present disclosure may be expressed using, for example, the expression vector of the present disclosure. A method for expressing the material protein of the present disclosure is not particularly limited and a known method can be employed. For example, a host may be used, or a cell-free protein synthesis system may be used.

In the former case, it is preferable to, for example, use the host into which the material protein of the present disclosure or the nucleic acid encoding the material protein has been introduced and culture the host to express the material protein of the present disclosure in the host. Thus, for example, introducing a nucleic acid encoding the material protein of the present disclosure into a host makes it possible to produce a transformant that synthesizes the material protein of the present disclosure, and the material protein of the present disclosure can be synthesized by culturing the transformant.

The method for culturing the host is not particularly limited and can be determined as appropriate in accordance with the type of the host. A culture medium for use in the culture is not particularly limited and can be determined as appropriate in accordance with the type of the host.

In the latter case, it is preferable to express the polynucleotide for the material protein of the present disclosure in a cell-free protein synthesis system. In this case, an expression vector may be used to express the polynucleotide for the material protein of the present disclosure. The cell-free protein synthesis system is implemented through a known method using, for example, a cell extract, a buffer containing various components, and an expression vector into which a polynucleotide encoding the material protein of the present disclosure is introduced, and for example, a commercially available reagent kit can be used.

The method for producing a protein according to the present disclosure may include, for example, a collection step of collecting the material protein of the present disclosure. The material protein of the present disclosure obtained in the collection step may be, for example, a crude product or a purified protein.

When the material protein is collected from the culture solution, insoluble matter is removed in the collection step by subjecting the culture supernatant to, for example, filtration, centrifugation, or the like. Then, in the collection step, the material protein of the present disclosure can be obtained by, for example, subjecting the culture supernatant from which the insoluble matter have been removed to separation and purification using the following techniques in combination as appropriate: concentration with an ultrafilter; salting-out such as ammonium sulfate precipitation; dialysis; and chromatography with various columns such as an ion-exchange column and a gel-filtration column.

When the material protein is collected from the transformant, for example, the transformant is homogenized through pressurization, ultrasonication, or the like in the collection step. The material protein of the present disclosure can be obtained by removing insoluble matter from the obtained homogenate solution and subjecting the resulting solution to separation and purification as described above.

For example, the material protein of the present disclosure obtained through the production method of the present disclosure may be used as a crude protein as it is, or may be partially purified and used as a partially purified protein, or may be purified to a single product and used as a purified protein.

In the production method of the present disclosure, powder of the obtained material protein of the present disclosure may be produced through, for example, freeze drying, vacuum drying, or spray drying. In this case, in the production method of the present disclosure, for example, the protein of the present invention may be dissolved in a buffer solution such as an acetate buffer solution, a phosphate buffer solution, a triethanolamine buffer solution, a tris-hydrochloric acid buffer solution, a GOOD's buffer (e.g., HEPES, PIPES, MES, MOPS, or the like), or the like in advance.

### EXAMPLES

Hereinafter, the present disclosure will be described in detail by way of examples, but the present disclosure is not limited to aspects described in the examples.

### Example 1

It was confirmed that a heteromultimeric protein could be produced using the production method of the present disclosure.

### (1) Preparation of Plasmid Vector

First, it was confirmed whether a first complex that included two proteins for forming a heterodimer could be formed using the production method of the present disclosure. Specifically, plasmid vectors capable of expressing four proteins below were constructed.
First protein: a protein (VHH-SpyTag-CD3 L chain) that includes a solubilization domain, SpyTag (binding tag), and a CD3 antibody light chain
Second protein: a protein (VHH-SpyCatcher-CD3 H chain) that includes a solubilization domain, SpyCatcher (binding partner), a CD3 antibody heavy chain, and a CD3 antibody variable domain
Third protein: a Herceptin antibody light chain
Fourth protein: a Herceptin antibody heavy chain

A plasmid vector capable of expressing the VHH-SpyTag-CD3 L chain was constructed in accordance with the following procedure. First, the entire length of a synthetic gene (Eurofins Genomics K.K.) that included base sequences encoding a signal peptide, aGFP4 (single-chain antibody (solubilization domain), SEQ ID NO: 9), a G1 linker, SpyTag (SEQ ID NO: 2), a thrombin cleavage sequence, a light chain variable region of M291, and a light chain constant region of M291 was amplified using PCR. The obtained full-length synthetic gene was linked to an animal cell expression vector (pCDNA3.4) to construct a recombinant protein expression vector. In the expression vector, as the VHH-SpyTag-CD3 L chain region (SEQ ID NO: 10), the signal peptide, the aGFP4, the G1 linker, the SpyTag, the thrombin cleavage sequence, the light chain variable region of M291, and the light chain constant region of M291 were linked in this order from the N terminus to the C terminus as indicated using brackets.

(SEQ ID NO: 9)
aGFP4
QVQLVESGGALVQPGGSLRLSCAASGFPVNRYSMRWYRQAPGKER
EWVAGMSSAGDRSSYEDSVKGRFTISRDDARNTVYLQMNSLKPED
TAVYYCNVNVGFEYWGQGTQVTVSS
VHH-Spy Tag-CD3 L chain region
(SEQ ID NO: 10)
[MEFGLSWLFLVAILKGVQC][QVQLVESGGALVQPGGSLRLSCA
ASGFPVNRYSMRWYRQAPGKEREWVAGMSSAGDRSSYEDSVKGRF
TISRDDARNTVYLQMNSLKPEDTAVYYCNVNVGFEYWGQGTQVTV
SS][GGSGG][AHIVMVDAYKPTK][GGSGGGGSGG][LVPRGSH
MHM][DIVLTQSPAIMSASPGEKVTMTCSASSSVSYMNWYKQKSG
TSPKRWTYDTSKLASGVPARFSGSGSGTSYSLTISSMEAEDAATY
YCQQWSSNPPTFGSGTKLEI][KRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC]

A plasmid vector capable of expressing the VHH-SpyCatcher-CD3 H chain was constructed in accordance with the following procedure. First, the entire length of a synthetic gene (Eurofins Genomics K.K.) that included base sequences encoding a signal peptide, Ia1 (single-chain antibody (solubilization domain), SEQ ID NO: 11), a G1 linker, SpyCatcher (SEQ ID NO: 1), a G2 linker, a thrombin cleavage sequence, a heavy chain variable region of M291, a heavy chain constant region of M291, a hinge region, and an Fc region was amplified using PC& The obtained full-length synthetic gene was linked to an animal cell expression vector (pCDNA3.4) to construct a recombinant protein expression vector. In the expression vector, as the VHH-SpyCatcher-CD3 H chain region (SEQ ID NO: 12), the signal peptide, the Ia1, the G1 linker, the SpyCatcher, the G2 linker, the thrombin cleavage sequence, the heavy chain variable region of M291, the heavy chain constant region of M291, the hinge region, and the Fc region were linked in this order from the N terminus to the C terminus as indicated using brackets.

la1
(SEQ ID NO: 11)
QVQLQESGGGLVQAGGSLLLSCAASGRTFSSYAMGWFRQAPGKER
EFVAAINWSGGSTSYADSVKGRFTISRDNTKNTVYLQMNSLKPED
TAAFYCAATYNPYSRDHYFPRMTTEYDYWGQGTQVTVSS
VHH-SpyCatcher-CD3 H chain region
(SEQ ID NO: 12)
[MEFGLSWLFLVAILKGVQC][QVQLQESGGGLVQAGGSLLLSCAA
SGRTFSSYAMGWFRQAPGKEREFVAAINWSGGSTSYADSVKGRFTI
SRDNTKNTVYLQMNSLKPEDTAAFYCAATYNPYSRDHYFPRMTTEY
DYWGQGTQVTVSS][GGSGG][DSATHIKFSKRDEDGKELAGATM
ELRDSSGKTISTWISDGQVKDFYLYPGKYTFVETAAPDGYEVATA
ITFTVNEQGQVTVNG][GGSGGGGSGG][LVPRGSHMHM][QVQL
QQSGAELARPGASVKMSCKASGYTFISYTMHWVKQRPGQGLEWIG
YINPRSGYTHYNQKLKDKATLTADKSSSSAYMQLSSLTSEDYAVY
YCARSAYYDYDGFAYWGQGTLVTVSA][ASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKV][EPKSCDK
TH][TCPPCP][APELLGGPSVFLFPPKPKDTLMISRTPEVTCVV
VDVSHEDPEVKFNWYVDGVEVHNAKTKPREEQYNSTYRVVSVLTV
LHQDWLNGKEYKCKVSNKALPAPIEKTISKAKGQPREPQVCTLPP
SRDELTKNQVSLSCAVKGFYPSDIAVEWESNGQPENNYKTTPPVL
DSDGSFFLVSKLTVDKSRWQQGNVFSCSVMHEALHNHYTQKSLSL
SPGK]

A plasmid vector capable of expressing the Herceptin antibody light chain was constructed in accordance with the following procedure. First, the entire length of a synthetic gene (Eurofins Genomics K.K.) that included base sequences encoding a signal peptide, a light chain variable region of h4D5, and a light chain constant region of h4D5 was amplified using PCR. The obtained full-length synthetic gene was linked to an animal cell expression vector (pCAGGS) to construct a recombinant protein expression vector. In the expression vector, as the Herceptin antibody light chain region (SEQ ID NO: 13), the signal peptide, the light chain variable region of h4D5, and the light chain constant region of h4D5 were linked in this order from the N terminus to the C terminus as indicated using brackets.

Herceptin antibody light chain region
(SEQ ID NO: 13)
[METPAQLLFLLLLWLPESTG][DIQMTQSPSSLSASVGDRVTIT
CRASQDVNTAVAWYQQKPGKAPKLLIYSASFLYSGVPSRFSGSRS
GTDFTLTISSLOPEDFATYYCQQHYTTPPTFGQGTKVEI][KRTV
AAPSVFIFPPSDEQLKSGTASVVCLLNNFYPREAKVQWKVDNALQ
SGNSQESVTEQDSKDSTYSLSSTLTLSKADYEKHKVYACEVTHQG
LSSPVTKSFNRGEC]

A plasmid vector capable of expressing the Herceptin antibody heavy chain was constructed in accordance with the following procedure.

First, the entire length of a synthetic gene (Eurofins Genomics K.K.) that included base sequences encoding a signal peptide, a heavy chain variable region of h4D5, a heavy chain constant region of h4D5, a hinge region, and an Fc region of h4D5 was amplified using PCR. The obtained full-length synthetic gene was linked to an animal cell expression vector (pCAGGS) to construct a recombinant protein expression vector. In the expression vector, as the Herceptin antibody heavy chain region (SEQ ID NO: 14), the signal peptide, the heavy chain variable region of h4D5, the heavy chain constant region of h4D5, the hinge region, and the Fc region of h4D5 were linked in this order from the N terminus to the C terminus as indicated using brackets.

Herceptin antibody heavy chain region
(SEQ ID NO: 14)
[MEFGLSWLFLVAILKGVQC][EVOLVESGGGLVQPGGSLRLSCA
ASGFNIKDTYIHWVRQAPGKGLEWVARIYPTNGYTRYADSVKGRF
TISADTSKNTAYLQMNSLRAEDTAVYYCSRWGGDGFYAMDYWGQG
TLVTVSS][ASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKKV][EPKSCDKTH][TCPPCP][APELLGG
PSVFLFPPKPKDTLMISRTPEVTCVVVDVSHEDPEVKFNWYVDGV
EVHNAKTKPREEQYNSTYRVVSVLTVLHQDWLNGKEYKCKVSNKA
LPAPIEKTISKALGQPREPQVYTLPPCRDELTKNQVSLWCLVKGF
YPSDIAVEWESNGQPENNYKTTPPVLDSDGSFFLYSKLTVDKSRW
QQGNVFSCSVMHEALHNHYTQKSLSLSPGK]

Next, Expi293F cells were transfected with the four recombinant protein plasmid expression vectors. First, frozen Expi293F cells were thawed and seeded in an HE400 culture media. After seeded, the cells were cultured at 37° C. at 8% CO₂ with shaking at 125 rpm until the number of the cells reached 3 to 5×10⁶/ml and the viability reached 95% or more. After the culture, the culture solution was added to 25 ml of the HE400 culture medium such that the number of the cells was 75×10⁶ cells. After the addition, the plasmid vectors were added to Opti-MEM (registered trademark) such that the final concentration of the total amount thereof was 30000 ng/ml. In parallel with this, separately from the Opti-MEM (registered trademark) to which the plasmid vectors had been added, PEI was added to Opti-MEM (registered trademark) such that the final concentration was 40 μg/ml. After the addition, each Opti-MEM (registered trademark) was left to stand at room temperature (24° C.; the same applies hereinafter) for 5 minutes. After the standing, the Opti-MEM (registered trademark) containing the plasmid vectors and the Opti-MEM (registered trademark) containing PEI were mixed, and the resulting mixture was left to stand at room temperature for 20 minutes. After the standing, the mixture was added to 25 ml of the HE400 culture medium, and the cells were cultured at 37° C. at 8% CO₂ with shaking at 125 rpm for 20 hours. After the culture, sodium valproate (final concentration: 1.25 μmol/l), sodium propionate (final concentration: 4 μmol/l), and 750 μl of 20 wt % tryptone were added. After the addition, the cells were cultured at 37° C. at 8% CO₂ with shaking at 125 rpm for 6 days. After the culture, the culture medium supernatant was collected.

### (2) Purification of Protein

The culture supernatant was loaded into a protein A column (manufactured by SUPrA or Bio-Rad Laboratories, Inc.) with the column volume set to 1 ml per 30-ml culture. Then, a flow-through fraction was collected. Subsequently, a wash buffer (50 mmol/l sodium phosphate buffer solution) was loaded into the column, and a wash fraction was collected. After the collection, an elute buffer (20 mmol/l sodium citrate+100 mmol/l sodium chloride (pH 3.0)) was loaded, and an elution fraction was collected. The elution fraction collected was poured into a dialysis membrane, and was dialyzed against a dialysis buffer (150 mmol/l NaCl, 50 mmol/l HEPES). After the dialysis, the sample was concentrated using a concentration tube to obtain a purified sample.

### (3) Examination of Purified Protein

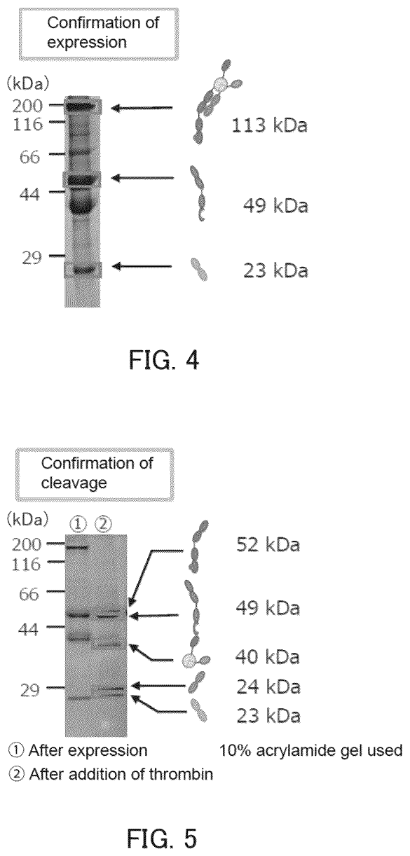

It was examined using SDS-PAGE whether a complex that included the VHH-SpyTag-CD3 L chain and the VHH-SpyCatcher-CD3 H chain was formed in the purified sample obtained in Example 1(2) above. Specifically, 10 μl of a 5×SDS buffer was added to the purified sample (40 μl) to obtain a suspension. After the suspension, heat treatment was conducted at 95° C. for 5 minutes. After the heat treatment, the sample was applied in the wells of 10% polyacrylamide gel, and electrophoresis was conducted at 150 to 200 V. A marker and the purified sample were applied on the 10% polyacrylamide gel. After the electrophoresis, the polyacrylamide gel was stained with a CBB (Coomassie Brilliant Blue) staining solution for 5 minutes, and destained with a destaining solution. FIG. 4 shows the results.

FIG. 4 is a photograph illustrating the result of the SDS-PAGE. In FIG. 4, the molecule weight (kDa) is shown on the left side of the photograph. As shown in FIG. 4, in the purified sample, a band was detected at the estimated molecular weight of the complex. Also, bands were detected at the estimated molecular weights of the Herceptin antibody light chain and the Herceptin antibody heavy chain. These results suggested that the complex that included the two proteins could be formed using the production method of the present disclosure.

### (4) Examination of Complex Through Cleavage by Thrombin

Next, it was examined whether the band at the estimated molecular weight of the complex in Example 1(3) above was derived from the complex that included the VHH-SpyTag-CD3 L chain and the VHH-SpyCatcher-CD3 H chain. Specifically, the protein was purified in the same manner as in Example 1(2) above, and the elution fraction after the purification was added to a thrombin buffer (2.5 mmol/l CaCl₂), 150 mmol/l NaCl, 20 mmol/l Tris-HCl (pH8.0)). After the addition, the resulting mixture was dialyzed for 6 hours. Then, thrombin (cleaving ability: 2 unit/μl, manufactured by Wako Pure Chemical Industries, Ltd.) was added in accordance with the yield of the protein. After the addition, the mixture was left to stand at 25° C. overnight. After the standing, a sample after the cleavage reaction was purified using a protein A column. After the purification, a purified thrombin-cleaved sample was obtained. Then, SDS-PAGE was conducted. The SDS-PAGE was conducted in the same manner as in Example 1(3) above, except that the purified thrombin-cleaved sample was used as a sample for the SDS-PAGE in addition to the purified sample. FIG. 5 shows the results.

FIG. 5 is a photograph illustrating the result of the SDS-PAGE. In FIG. 5, the types of samples are shown on the upper side of the photograph, and the molecular weight (kDa) is shown on the left side of the photograph. As shown in FIG. 5, in the purified thrombin-cleaved sample, no band was detected at the estimated molecular weight of the complex. Also, as shown in FIG. 5, in the purified thrombin-cleaved sample, bands were detected at the estimated molecular weights of the Herceptin antibody light chain, the Herceptin antibody heavy chain, the CD3 antibody light chain, the CD3 antibody heavy chain, and the VHH-SpyTag and the VHH-SpyCatcher binding together. It was found from these results that the complex that included the two proteins could be formed using the production method of the present disclosure. Also, it was found that, in the complex, the cleavable domains were cleaved by thrombin.

### (5) Separation of Herceptin Through Chromatography

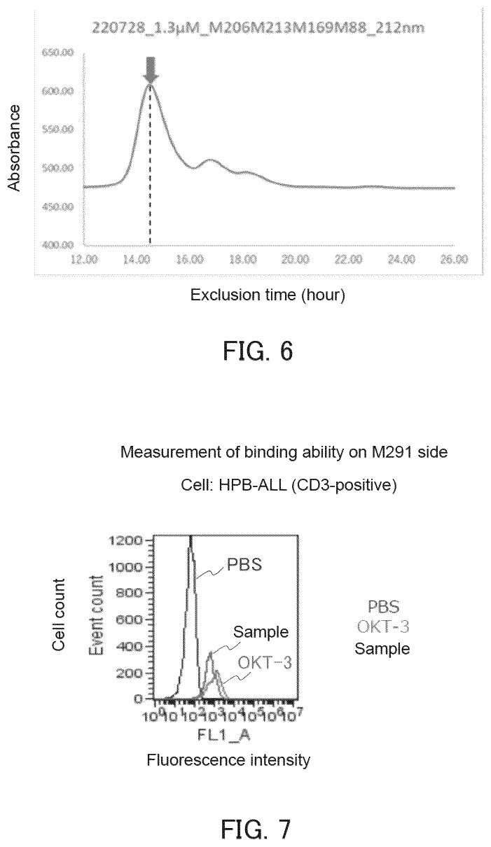

Herceptin was separated from the purified sample purified in Example 1(3) above using size-exclusion chromatography. Specifically, the protein purified in Example 1(3) above was loaded into a gel filtration chromatography column (Superdex 200 increase 30/100 GL, manufactured by GE HealthCare) at 0.5 mL/min, and the absorbance at 212 nm was measured at room temperature (about 25° C.). In the measurement above, 1×PBS was used as a buffer. FIG. 6 shows the results.

FIG. 6 is a graph illustrating a Herceptin elution pattern of the size-exclusion chromatography. In FIG. 6, the horizontal axis indicates the exclusion time (hour), and the vertical axis indicates the absorbance. As shown in FIG. 6, the elution of Herceptin obtained using the production method of the present disclosure was confirmed (arrow in FIG. 6).

### (6) Evaluation of Activity Against CD3-Positive Cell

It was examined using flow cytometry whether a CD3 antibody light chain and a CD3 antibody heavy chain obtained in the same manner as in Example 1(5) above bound to a CD3-expressing cell. Specifically, an HPB-ALL strain overexpressing CD3 on the cell membrane surface was used, and measurement was conducted using a flow cytometer. An equal amount of the HPB-ALL that had been cultured in a T75 flask was transferred to two 15-ml Falcon tubes and centrifuged at 1500 rpm for 5 minutes. After the centrifugation, the supernatant was aspirated using an aspirator, and a necessary amount of 1×PBS was added to obtain a suspension. A cell suspension of 1×10⁶ cells/ml was thus obtained. Thereafter, 1 ml of the suspension was dispensed into three microtubes (a) to (c) and diluted with 1×PBS. After the dilution, the two microtubes (a) and (b) were centrifuged at 2000 rpm at 25° C. for 7 minutes. After the centrifugation, the supernatant was aspirated using an aspirator, and 1 ml of 1×PBS was added. After the addition, the two microtubes (a) and (b) were centrifuged again at 2000 rpm at 25° C. for 7 minutes. After the centrifugation, the supernatant was aspirated using an aspirator, 1×PBS and the CD3 antibody purified in the same manner as in Example 1(5) above were added to one of the microtubes, microtube (b), such that the final concentration of the CD3 antibody was 0.05 μmol/l, followed by inversion mixing. After the inversion mixing, the microtube was left to stand for 20 minutes. After the standing, the tube was centrifuged at 2000 rpm at 25° C. for 7 minutes. Thereafter, 1 μl of OKT3-FITC (manufactured by COSMO BIO Co., Ltd.) and 1 ml of 1×PBS were added to the microtube (a) and 1 μl of anti-Fc-FITC (manufactured by Abcam Limited) and 1 ml of 1×PBS were added to the microtube (b), followed by inversion mixing. After the inversion mixing, the microtubes were left to stand for 20 minutes. After the standing, the tubes were centrifuged at 2000 rpm at 25° C. for 7 minutes. After the centrifugation, the supernatant was aspirated using an aspirator, and 1 ml of 1×PBS was added. After the addition, the tubes were centrifuged again at 2000 rpm at 25° C. for 7 minutes. After the centrifugation, the supernatant was aspirated using an aspirator, and 1 ml of 1×PBS was added to obtain a suspension. After the suspension, the obtained suspensions were sterilized using a mesh filter. After the measurement conditions were set, a cell analyzer RF-500 (manufactured by Sysmex Corporation) was used to measure the negative control (c), the positive control (a), and the sample (b) in this order. After the measurement, FCSalyzer was used to show the measurement results graphically. FIG. 7 shows the results.

FIG. 7 is a graph illustrating binding of the CD3 antibody light chain and the CD3 antibody heavy chain to CD3-positive cells measured through flow cytometry. In FIG. 7, the horizontal axis indicates the fluorescence intensity, and the vertical axis indicates the cell count. As shown in FIG. 7, it was found that, when the CD3 antibody light chain and the CD3 antibody heavy chain obtained using the production method of the present disclosure were added to CD3-positive cells, the fluorescent intensity was enhanced compared with the case where they were not added to CD3-positive cells. It was found from these results that the CD3 antibody light chain and the CD3 antibody heavy chain obtained using the production method of the present disclosure bound to CD3-positive cells.

### (7) Evaluation of Activity Against HER2-Positive Cell

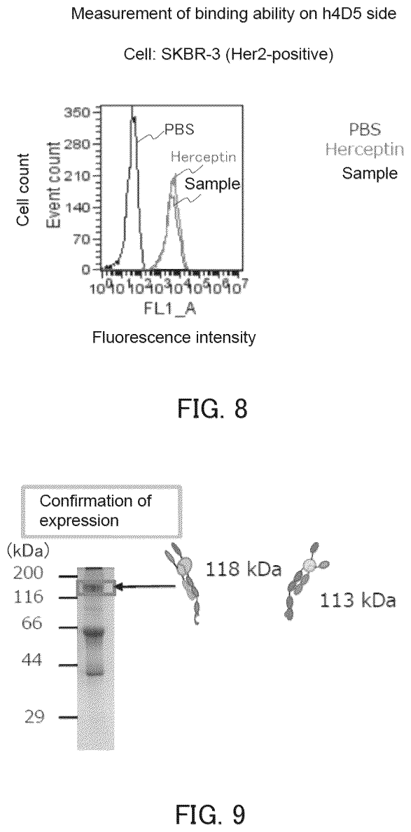

It was examined using flow cytometry whether the Herceptin antibody light chain and the Herceptin antibody heavy chain obtained in Example 1(5) above bound to a breast cancer cell. Specifically, an SK-BR-3 strain overexpressing HER2 on the cell membrane surface was used, and measurement was conducted using a flow cytometer. The supernatant of the SK-BR-3 that had been cultured in a T75 flask was aspirated using an aspirator, and a necessary amount of 1×PBS was added to obtain a cell suspension. Thereafter, 1 ml of the suspension containing 1×10⁶ cells was dispensed into three microtubes (d) to (f) and diluted with 1×PBS. After the dilution, the two microtubes (d) and (e) were centrifuged at 2000 rpm at 25° C. for 7 minutes. After the centrifugation, the supernatant was aspirated using an aspirator, and 1 ml of 1×PBS was added. After the addition, the two microtubes (d) and (e) were centrifuged again at 2000 rpm at 25° C. for 7 minutes. After the centrifugation, the supernatant was aspirated using an aspirator. After the aspiration, 1 μl of 5 mg/ml Herceptin (manufactured by Chugai Pharmaceutical Co., Ltd.) and 1 ml of 1×PBS were added to one of the microtubes, microtube (d), followed by inversion mixing. In parallel with this, 1 ml of 1×PBS and the Herceptin antibody purified in Example 1(5) above were added to one of the microtubes, microtube (e), such that the final concentration of the Herceptin antibody was 0.1 μmol/l, followed by inversion mixing. After the inversion mixing, the two microtubes (d) and (e) were left to stand for 20 minutes. After the standing, the two microtubes (d) and (e) were centrifuged at 2000 rpm at 25° C. for 7 minutes. After the centrifugation, the supernatant was aspirated using an aspirator, and 1 ml of 1×PBS was added. After the addition, the two microtubes (d) and (e) were centrifuged again at 2000 rpm at 25° C. for 7 minutes. After the centrifugation, the supernatant was aspirated using an aspirator, and 1 μl of anti-Fc-FITC (manufactured by Abcam Limited) and 1 ml of 1×PBS were added to the two microtubes (d) and (e), followed by inversion mixing. After the inversion mixing, the microtubes were left to stand for 20 minutes. After the standing, the two microtubes were centrifuged at 2000 rpm at 25° C. for 7 minutes. After the centrifugation, the supernatant was aspirated using an aspirator, and 1 ml of 1×PBS was added. After the addition, the two microtubes (d) and (e) were centrifuged again at 2000 rpm at 25° C. for 7 minutes. After the centrifugation, the supernatant was aspirated using an aspirator, and 1 ml of 1×PBS was added to obtain a suspension. After the suspension, the obtained suspensions were sterilized using a mesh filter. After the measurement conditions were set, a cell analyzer RF-500 (manufactured by Sysmex Corporation) was used to measure the negative control (f), the positive control (d), and the sample (e) in this order. After the measurement, FCSalyzer was used to show the measurement results graphically. FIG. 8 shows the results.

FIG. 8 is a graph illustrating binding of the Herceptin antibody light chain and the Herceptin antibody heavy chain to HER2-positive cells measured through flow cytometry. In FIG. 8, the horizontal axis indicates the fluorescence intensity, and the vertical axis indicates the cell count. As shown in FIG. 8, it was found that, when the Herceptin antibody light chain and the Herceptin antibody heavy chain obtained using the production method of the present disclosure were added to HER2-positive cells, the fluorescent intensity was enhanced compared with the case where they were not added to HER2-positive cells. It was found from these results that the Herceptin antibody light chain and the Herceptin antibody heavy chain obtained using the production method of the present disclosure bound to Her2-positive breast cancer cells.

### (8) Examination of Formation of Two Complexes

Since it was found that a complex that includes two proteins can be produced using the production method of the present disclosure, it was confirmed that the Herceptin antibody light chain and the Herceptin antibody heavy chain could also form a complex in the same manner as in Example 1(1) above. Specifically, plasmid vectors capable of expressing four proteins below for forming a heterotetramer were constructed. Plasmid vectors for a first protein and a second protein were constructed in the same manner as in Example 1(1) above.
First protein: a protein (VHH-SpyTag-CD3 L chain) that includes a variable domain, SpyTag (binding tag), and a CD3 antibody light chain
Second protein: a protein (VHH-SpyCatcher-CD3 H chain) that includes SpyCatcher (binding partner), a CD3 antibody heavy chain, and a CD3 antibody variable domain
Fifth protein: a protein (VHH-SnoopTag-Herceptin L chain) that includes a variable domain, SnoopTag (binding tag), and a Herceptin antibody light chain
Sixth protein: a protein (VHH-SnoopCatcher-Herceptin H chain) that includes SnoopCatcher (binding partner), a Herceptin antibody heavy chain, and a Herceptin variable domain.

A plasmid vector capable of expressing the VHH-SnoopTag-Herceptin L chain was constructed in accordance with the following procedure. First, the entire length of a synthetic gene (Eurofins Genomics K.K.) that included base sequences encoding a signal peptide, aGFP4 (single-chain antibody (solubilization domain), SEQ ID NO: 9), a G1 linker, SnoopTag (SEQ ID NO: 4), a G2 linker, a thrombin cleavage sequence, a light chain variable region of H4D5, and a light chain constant region of H4D5 was amplified using PCR. The obtained full-length synthetic gene was linked to an animal cell expression vector (pCAGEN) to construct a recombinant protein expression vector. In the expression vector, as the VHH-SnoopTag-Herceptin L chain region (SEQ ID NO: 15), the signal peptide, the aGFP4, the G1 linker, the SnoopTag, the G2 linker, the thrombin cleavage sequence, the light chain variable region of H4D5, and the light chain constant region of H4D5 were linked in this order from the N terminus to the C terminus as indicated using brackets.

VHH-SnoopTag-Herceptin L chain region
(SEQ ID NO: 15)
[METPAQLLFLLLLWLPESTG][QVQLVESGGALVQPGGSLRLSC
AASGFPVNRYSMRWYRQAPGKEREWVAGMSSAGDRSSYEDSVKGR
FTISRDDARNTVYLQMNSLKPEDTAVYYCNVNVGFEYWGQGTQVT
VSS][GGSGG][KLGDIEFIKVNK][GGSGGGGSGG][LVPRGSH
MHM][DIQMTQSPSSLSASVGDRVTITCRASQDVNTAVAWYQQKP
GKAPKLLIYSASFLYSGVPSRFSGSRSGTDFTLTISSLQPEDFAT
YYCQQHYTTPPTFGQGTKVEI][KRTVAAPSVFIFPPSDEQLKSG
TASVVCLLNNFYPREAKVQWKVDNALQSGNSQESVTEQDSKDSTY
SLSSTLTLSKADYEKHKVYACEVTHQGLSSPVTKSFNRGEC]

A plasmid vector capable of expressing the VHH-SnoopCatcher-Herceptin H chain was constructed in accordance with the following procedure. First, the entire length of a synthetic gene (Eurofins Genomics K.K.) that included base sequences encoding a signal peptide, the Ia1 (SEQ ID NO: 11) described above, a G1 linker, SnoopCatcher (SEQ ID NO: 3), a G2 linker, a thrombin cleavage sequence, a heavy chain variable region of H4D5, a heavy chain constant region of H4D5, a hinge region, and an Fc region was amplified using PCR. The obtained full-length synthetic gene was linked to an animal cell expression vector (pCAGGS) to construct a recombinant protein expression vector. In the expression vector, as the VHH-SnoopCatcher-Herceptin H chain region (SEQ ID NO: 16), the signal peptide, the Ia1, the G1 linker, the SnoopCatcher, the G2 linker, the thrombin cleavage sequence, the heavy chain variable region of H4D5, the heavy chain constant region of H4D5, the hinge region, and the Fc region were linked in this order from the N terminus to the C terminus as indicated using brackets.

VHH-SnoopCatcher-Herceptin H chain region
(SEQ ID NO: 16)
[MEFGLSWLFLVAILKGVQ][CQVQLQESGGGLVQAGGSLLLSCA
ASGRTFSSYAMGWFRQAPGKEREFVAAINWSGGSTSYADSVKGRF
TISRDNTKNTVYLQMNSLKPEDTAAFYCAATYNPYSRDHYFPRMT
TEYDYWGQGTQVTVSS][GGSGG][KPLRGAVFSLQKQHPDYPDI
YGAIDQNGTYQNVRTGEDGKLTFKNLSDGKYRLFENSEPAGYKPV
QNKPIVAFQIVNGEVRDVTSIVPQDIPATYEFTNDKHYITNEPIP
PK][GGSGGGGSGG][LVPRGSHMHM][EVQLVESGGGLVQPGGS
LRLSCAASGFNIKDTYIHWVRQAPGKGLEWVARIYPTNGYTRYAD
SVKGRFTISADTSKNTAYLQMNSLRAEDTAVYYCSRWGGDGFYAM
DYWGQGTLVTVSS][ASTKGPSVFPLAPSSKSTSGGTAALGCLVK
DYFPEPVTVSWNSGALTSGVHTFPAVLQSSGLYSLSSVVTVPSSS
LGTQTYICNVNHKPSNTKVDKKV][EPKSCDKTH][TCPPCP][A
PELLGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALPAPIEKTISKALGQPREPQVYTLPPCRDELTKNQVSLW
CLVKGFYPSDIAVEWESNGQPENNYKTTPPVLDSDGSFFLYSKLT
VDKSRWQQGNVFSCSVMHEALHNHYTQKSLSLSPGK]

Next, in the same manner as in Example 1(1), the four plasmid expression vectors for the recombinant proteins were transfected, the cells were cultured, and the supernatant of the culture medium was collected. After the collection, the protein was purified in the same manner as in Example 1(2) to obtain a purified sample. It was examined using SDS-PAGE whether a complex that included the VHH-SpyTag-CD3 L chain and the VHH-SpyCatcher-CD3 H chain and a complex that included the VHH-SnoopTag-Herceptin L chain and the VHH-SnoopCatcher-Herceptin H chain were formed in the purified sample. Specifically, 10 μl of a 5×SDS buffer was added to the purified sample (40 μl) to obtain a suspension. After the suspension, heat treatment was conducted at 95° C. for 5 minutes. After the heat treatment, the sample was applied in the wells of 10% polyacrylamide gel, and electrophoresis was conducted at 150 to 200 V. A marker and the purified sample were applied on the 10% polyacrylamide gel. After the electrophoresis, the polyacrylamide gel was stained with a CBB (Coomassie Brilliant Blue) staining solution for 5 minutes, and destained with a destaining solution. FIG. 9 shows the results.

FIG. 9 is a photograph illustrating the result of the SDS-PAGE. In FIG. 9, the molecule weight (kDa) is shown on the left side of the photograph. As shown in FIG. 9, in the purified sample, bands were detected at the estimated molecular weights of the two complexes. These results suggested that the two complexes could be formed using the production method of the present disclosure.

### (9) Examination of Two Complexes Through Cleavage by Thrombin

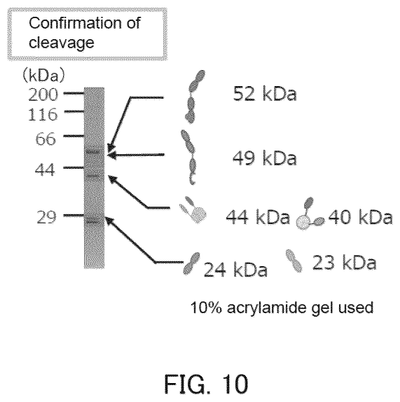

It was examined whether the bands at the estimated molecular weights of the two complexes in Example 1(8) above were derived from the complex that included the VHH-SpyTag-CD3 L chain and the VHH-SpyCatcher-CD3 H chain and the complex that included the VHH-SnoopTag-Herceptin L chain and the VHH-SnoopCatcher-Herceptin H chain. Specifically, the examination was conducted in the same manner as in Example 1(4), except that the complex that included the VHH-SnoopTag-Herceptin L chain and the VHH-SnoopCatcher-Herceptin H chain was used as a sample in addition to the complex that included the VHH-SpyTag-CD3 L chain and the VHH-SpyCatcher-CD3 H chain. FIG. 10 shows the results.

FIG. 10 is a photograph illustrating the result of the SDS-PAGE. In FIG. 10, the molecule weight (kDa) is shown on the left side of the photograph. As shown in FIG. 10, in the purified thrombin-cleaved sample, no bands were detected at the estimated molecular weights of the complex that included the VHH-SpyTag-CD3 L chain and the VHH-SpyCatcher-CD3 H chain and the complex that included the VHH-SnoopTag-Herceptin L chain and the VHH-SnoopCatcher-Herceptin H chain. Also, as shown in FIG. 10, in the purified thrombin-cleaved sample, bands were detected at the estimated molecular weights of the Herceptin antibody light chain, the Herceptin antibody heavy chain, the CD3 antibody light chain, the CD3 antibody heavy chain, the VHH-SpyTag and the VHH-SpyCatcher binding together, and the VHH-SnoopTag and the VHH-SnoopCatcher binding together. It was found from these results that the two complexes could be formed using the production method of the present disclosure. Also, it was found that, in the two complexes, the cleavable domains were cleaved by thrombin.

As described above, the present invention has been described with reference to the embodiments, but the present invention is not limited to the above-described embodiments. Various modifications that can be understood by a person skilled in the art can be made in the configurations and details of the present invention without departing from the scope of the present invention.

The present application claims the benefit of priority from Japanese Patent Application No. 2022-158540 filed on Sep. 30, 2022, the entire disclosure of which is incorporated herein.

### SUPPLEMENTARY NOTES

Some or all of the embodiments and the examples given above can be described as in the following supplementary notes, but the scope of the present disclosure is not limited thereto.

### <Method for Producing Heteromultimeric Protein>

### Supplementary Note 1

A method for producing a heteromultimeric protein, including:
a complex formation step of forming a first complex of two proteins by bringing the two proteins into contact with each other,
   the two proteins including a first protein and a second protein,
   the first protein including a first binding tag, a first cleavable domain, and a first domain in this order from an N terminus to a C terminus,
   the second protein including a first binding partner capable of binding to the first binding tag, a second cleavable domain, and a second domain in this order from an N terminus to a C terminus,
   the first protein and the second protein being capable of forming a dimer via a binding between the first domain and the second domain, and
   the first complex being formed by the first binding tag and the first binding partner binding together and the first domain and the second domain binding together, and
a generation step of generating a heterodimer composed of the first domain and the second domain by cleaving the first cleavable domain and the second cleavable domain in the first complex.

### Supplementary Note 2

The production method according to Supplementary Note 1, wherein the first cleavable domain and the second cleavable domain are the same cleavable domain.

### Supplementary Note 3

The production method according to Supplementary Note 1 or 2, wherein the first cleavable domain and/or the second cleavable domain includes a cleavable sequence of a self-cleaving peptide and/or a sequence capable of being cleaved by a protease or peptidase.

### Supplementary Note 4

The production method according to Supplementary Note 3, wherein the sequence capable of being cleaved by a protease is a thrombin cleavage sequence.

### Supplementary Note 5

The production method according to any one of Supplementary Notes 1 to 4, wherein the first binding tag and the first binding partner are a peptide tag and a peptide capable of spontaneously forming a covalent bond.

### Supplementary Note 6

The production method according to any one of Supplementary Notes 1 to 5, wherein a combination of the first binding tag and the first binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 7

The production method according to any one of Supplementary Notes 1 to 6, wherein the binding between the first domain and the second domain is a disulfide bond.

### Supplementary Note 8

The production method according to any one of Supplementary Notes 1 to 7, further including a first expression step of allowing a host cell to express the first protein and the second protein prior to the complex formation step.

### Supplementary Note 9

The production method according to any one of Supplementary Notes 1 to 8, further including a first purification step of purifying the first complex after the complex formation step.

### Supplementary Note 10

The production method according to any one of Supplementary Notes 1 to 9, further including a second purification step of purifying the heterodimer after the generation step.

### Supplementary Note 11

The production method according to any one of Supplementary Notes 1 to 7,
wherein the complex formation step is a step of forming a second complex of four proteins by bringing the four proteins into contact with one another,
   the four proteins including the first protein, the second protein, a third protein, and a fourth protein,
   the third protein including a second binding tag, a third cleavable domain, and a third domain in this order from an N terminus to a C terminus,
   the fourth protein including a second binding partner capable of binding to the second binding tag, a fourth cleavable domain, and a fourth domain in this order from an N terminus to a C terminus,
   the second protein and the fourth protein being capable of forming a dimer via a binding between the second domain and the fourth domain,
   the third protein and the fourth protein being capable of forming a dimer via a binding between the third domain and the fourth domain, and
   the second complex being formed by the first binding tag and the first binding partner binding together, the second binding tag and the second binding partner binding together, the first domain and the second domain binding together, the second domain and the fourth domain binding together, and the third domain and the fourth domain binding together, and
in the generation step, a heterotetramer of the first domain, the second domain, the third domain, and the fourth domain is generated by cleaving the first cleavable domain, the second cleavable domain, the third cleavable domain, and the fourth cleavable domain in the second complex.

### Supplementary Note 12

The production method according to Supplementary Note 11, wherein the third cleavable domain and the fourth cleavable domain are the same cleavable domain.

### Supplementary Note 13

The production method according to Supplementary Note 11 or 12, wherein the third cleavable domain and/or the fourth cleavable domain includes a cleavable sequence of a self-cleaving peptide and/or a sequence capable of being cleaved by a protease or peptidase.

### Supplementary Note 14

The production method according to Supplementary Note 13, wherein the sequence capable of being cleaved by a protease is a thrombin cleavage sequence.

### Supplementary Note 15

The production method according to any one of Supplementary Notes 11 to 14, wherein the second binding tag and the second binding partner are a peptide tag and a peptide capable of spontaneously forming a covalent bond.

### Supplementary Note 16

The production method according to any one of Supplementary Notes 11 to 15, wherein a combination of the second binding tag and the second binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 17

The production method according to any one of Supplementary Notes 11 to 16, wherein the binding between the third domain and the fourth domain and/or the binding between the second domain and the fourth domain is a disulfide bond.

### Supplementary Note 18

The production method according to any one of Supplementary Notes 11 to 17,
wherein the first domain is a light chain of an antibody that binds to a first target,
the second domain is a heavy chain of the antibody that binds to the first target,
the third domain is a light chain of an antibody that binds to a second target, and
the fourth domain is a heavy chain of the antibody that binds to the second target.

### Supplementary Note 19

The production method according to Supplementary Note 18, wherein the antibody that binds to the first target and the antibody that binds to the second target recognize different epitopes.

### Supplementary Note 20

The production method according to Supplementary Note 18 or 19, wherein the antibody that binds to the first target and the antibody that binds to the second target recognize different antigens.

### Supplementary Note 21

The production method according to any one of Supplementary Notes 18 to 20, wherein the antibody that binds to the first target and the antibody that binds to the second target are IgG, IgA, IgE, IgD, or IgM.

### Supplementary Note 22

The production method according to Supplementary Note 21, wherein the IgG is IgG1, IgG2, IgG2a, IgG2b, IgG3, or IgG4.

### Supplementary Note 23

The production method according to any one of Supplementary Notes 11 to 22, wherein the first cleavable domain, the second cleavable domain, the third cleavable domain, and the fourth cleavable domain are the same cleavable domain.

### Supplementary Note 24

The production method according to any one of Supplementary Notes 11 to 23, wherein a combination of the first binding tag and the first binding partner and a combination of the second binding tag and the second binding partner are different from each other.

### Supplementary Note 25

The production method according to any one of Supplementary Notes 11 to 24,
wherein the second protein further includes a fifth cleavable domain and a third binding tag in this order at the C terminus,
the fourth protein further includes a sixth cleavable domain and a third binding partner capable of binding to the third binding tag in this order at the C terminus,
in the complex formation step, the third binding tag and the third binding partner also bind together, and
in the generation step, the fifth cleavable domain and the sixth cleavable domain in the second complex are cleaved.

### Supplementary Note 26

The production method according to Supplementary Note 25, wherein the fifth cleavable domain and the sixth cleavable domain are the same cleavable domain.

### Supplementary Note 27

The production method according to Supplementary Note 25 or 26, wherein the fifth cleavable domain and/or the sixth cleavable domain includes a cleavable sequence of a self-cleaving peptide and/or a sequence capable of being cleaved by a protease or peptidase.

### Supplementary Note 28

The production method according to Supplementary Note 27, wherein the sequence capable of being cleaved by a protease is a thrombin cleavage sequence.

### Supplementary Note 29

The production method according to any one of Supplementary Notes 25 to 28, wherein the third binding tag and the third binding partner are a peptide tag and a peptide capable of spontaneously forming a covalent bond.

### Supplementary Note 30

The production method according to any one of Supplementary Notes 25 to 29, wherein a combination of the third binding tag and the third binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 31

The production method according to any one of Supplementary Notes 25 to 30, wherein the first cleavable domain, the second cleavable domain, the third cleavable domain, the fourth cleavable domain, the fifth cleavable domain, and the sixth cleavable domain are the same cleavable domain.

### Supplementary Note 32

The production method according to any one of Supplementary Notes 25 to 31, wherein a combination of the first binding tag and the first binding partner, a combination of the second binding tag and the second binding partner, and a combination of the third binding tag and the third binding partner are different from one another.

### Supplementary Note 33

The production method according to any one of Supplementary Notes 11 to 32, further including a first expression step of allowing a host cell to express the first protein, the second protein, the third protein, and the fourth protein prior to the complex formation step.

### Supplementary Note 34

The production method according to any one of Supplementary Notes 11 to 33, further including a first purification step of purifying the second complex after the complex formation step.

### Supplementary Note 35

The production method according to any one of Supplementary Notes 11 to 34, further including a second purification step of purifying the heterodimer after the generation step.

### <Protein>

### Supplementary Note 36

A protein including a first binding tag capable of binding to a first binding partner, a first cleavable domain, and a first domain in this order from an N terminus to a C terminus.

### Supplementary Note 37

The protein according to Supplementary Note 36, wherein the first cleavable domain includes a cleavable sequence of a self-cleaving peptide and/or a sequence capable of being cleaved by a protease or peptidase.

### Supplementary Note 38

The protein according to Supplementary Note 37, wherein the sequence capable of being cleaved by a protease is a thrombin cleavage sequence.

### Supplementary Note 39

The protein according to any one of Supplementary Notes 36 to 38, wherein the first binding tag and the first binding partner are a peptide tag and a peptide capable of spontaneously forming a covalent bond.

### Supplementary Note 40

The protein according to any one of Supplementary Notes 36 to 39, wherein a combination of the first binding tag and the first binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 41

The protein according to any one of Supplementary Notes 36 to 40, wherein the first domain is a light chain of an antibody that binds to a first target.

### Supplementary Note 42

A protein including a first binding partner capable of binding to a first binding tag, a second cleavable domain, and a second domain in this order from an N terminus to a C terminus.

### Supplementary Note 43

The protein according to Supplementary Note 42, wherein the second cleavable domain includes a cleavable sequence of a self-cleaving peptide and/or a sequence capable of being cleaved by a protease or peptidase.

### Supplementary Note 44

The protein according to Supplementary Note 43, wherein the sequence capable of being cleaved by a protease is a thrombin cleavage sequence.

### Supplementary Note 45

The protein according to any one of Supplementary Notes 42 to 44, wherein the first binding tag and the first binding partner are a peptide tag and a peptide capable of spontaneously forming a covalent bond.

### Supplementary Note 46

The protein according to any one of Supplementary Notes 42 to 45, wherein a combination of the first binding tag and the first binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 47

The protein according to any one of Supplementary Notes 42 to 46, wherein the second domain is a heavy chain of an antibody that binds to a first target.

### Supplementary Note 48

The protein according to any one of Supplementary Notes 42 to 47, further including a fifth cleavable domain, and a third binding tag or a third binding partner capable of binding to the third binding tag in this order at the C terminus.

### Supplementary Note 49

The protein according to Supplementary Note 48, wherein the fifth cleavable domain includes a cleavable sequence of a self-cleaving peptide and/or a sequence capable of being cleaved by a protease or peptidase.

### Supplementary Note 50

The protein according to Supplementary Note 49, wherein the sequence capable of being cleaved by a protease is a thrombin cleavage sequence.

### Supplementary Note 51

The protein according to any one of Supplementary Notes 48 to 50, wherein the third binding tag and the third binding partner are a peptide tag and a peptide capable of spontaneously forming a covalent bond.

### Supplementary Note 52

The protein according to any one of Supplementary Notes 48 to 51, wherein the second cleavable domain and the fifth cleavable domain are the same cleavable domain.

### Supplementary Note 53

The protein according to any one of Supplementary Notes 48 to 52, wherein a combination of the first binding tag and the first binding partner and a combination of the third binding tag and the third binding partner are different combinations of a binding tag and a binding partner.

### Supplementary Note 54

The protein according to any one of Supplementary Notes 48 to 53, wherein a combination of the third binding tag and the third binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 55

A protein including a second binding tag capable of binding to a second binding partner, a third cleavable domain, and a third domain in this order from an N terminus to a C terminus.

### Supplementary Note 56

The protein according to Supplementary Note 55, wherein the third cleavable domain includes a cleavable sequence of a self-cleaving peptide and/or a sequence capable of being cleaved by a protease or peptidase.

### Supplementary Note 57

The protein according to Supplementary Note 56, wherein the sequence capable of being cleaved by a protease is a thrombin cleavage sequence.

### Supplementary Note 58

The protein according to any one of Supplementary Notes 55 to 57, wherein the second binding tag and the second binding partner are a peptide tag and a peptide capable of spontaneously forming a covalent bond.

### Supplementary Note 59

The protein according to any one of Supplementary Notes 55 to 58, wherein a combination of the second binding tag and the second binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 60

The protein according to any one of Supplementary Notes 55 to 59, wherein the third domain is a light chain of an antibody that binds to a second target.

### Supplementary Note 61

A protein including a second binding partner capable of binding to a second binding tag, a fourth cleavable domain, and a fourth domain in this order from an N terminus to a C terminus.

### Supplementary Note 62

The protein according to Supplementary Note 61, wherein the fourth cleavable domain includes a cleavable sequence of a self-cleaving peptide and/or a sequence capable of being cleaved by a protease or peptidase.

### Supplementary Note 63

The protein according to Supplementary Note 62, wherein the sequence capable of being cleaved by a protease is a thrombin cleavage sequence.

### Supplementary Note 64

The protein according to any one of Supplementary Notes 61 to 63, wherein the second binding tag and the second binding partner are a peptide tag and a peptide capable of spontaneously forming a covalent bond.

### Supplementary Note 65

The protein according to any one of Supplementary Notes 61 to 64, wherein a combination of the second binding tag and the second binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 66

The protein according to any one of Supplementary Notes 61 to 65, wherein the fourth domain is a heavy chain of an antibody that binds to a second target.

### Supplementary Note 67

The protein according to any one of Supplementary Notes 61 to 66, further including a sixth cleavable domain, and a third binding partner capable of binding to a third binding tag in this order at the C terminus.

### Supplementary Note 68

The protein according to Supplementary Note 67, wherein the sixth cleavable domain includes a cleavable sequence of a self-cleaving peptide and/or a sequence capable of being cleaved by a protease or peptidase.

### Supplementary Note 69

The protein according to Supplementary Note 68, wherein the sequence capable of being cleaved by a protease is a thrombin cleavage sequence.

### Supplementary Note 70

The protein according to any one of Supplementary Notes 67 to 69, wherein the third binding tag and the third binding partner are a peptide tag and a peptide capable of spontaneously forming a covalent bond.

### Supplementary Note 71

The protein according to any one of Supplementary Notes 67 to 70, wherein the fourth cleavable domain and the sixth cleavable domain are the same cleavable domain.

### Supplementary Note 72

The protein according to any one of Supplementary Notes 67 to 71, wherein a combination of the second binding tag and the second binding partner and a combination of the third binding tag and the third binding partner are different from each other.

### Supplementary Note 73

The protein according to any one of Supplementary Notes 67 to 72, wherein a combination of the third binding tag and the third binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 74

The protein according to any one of Supplementary Notes 36 to 73, for use in the method for producing a heteromultimeric protein according to any one of Supplementary Notes 1 to 35.

### <Heteromultimeric Protein>

### Supplementary Note 75

A protein including two proteins,
wherein the two proteins include a first protein and a second protein,
the first protein includes a first binding tag, a first cleavable domain, and a first domain in this order from an N terminus to a C terminus,
the second protein includes a first binding partner capable of binding to the first binding tag, a second cleavable domain, and a second domain in this order from an N terminus to a C terminus,
the first protein and the second protein form a dimer via a binding between the first domain and the second domain, and
the first binding tag and the first binding partner bind together.

### Supplementary Note 76

The protein according to Supplementary Note 75, wherein the first cleavable domain and the second cleavable domain are the same cleavable domain.

### Supplementary Note 77

The protein according to Supplementary Note 75 or 76, wherein the first cleavable domain and/or the second cleavable domain includes a cleavable sequence of a self-cleaving peptide and/or a sequence capable of being cleaved by a protease or peptidase.

### Supplementary Note 78

The protein according to any one of Supplementary Notes 75 to 77, wherein the first binding tag and the first binding partner are a peptide tag and a peptide capable of spontaneously forming a covalent bond.

### Supplementary Note 79

The protein according to any one of Supplementary Notes 75 to 78, wherein a combination of the first binding tag and the first binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 80

The production method according to any one of Supplementary Notes 75 to 79, wherein the binding between the first domain and the second domain is a disulfide bond.

### Supplementary Note 81

The protein according to any one of Supplementary Notes 75 to 80, further including a third protein and a fourth protein,
wherein the third protein includes a second binding tag, a third cleavable domain, and a third domain in this order from an N terminus to a C terminus,
the fourth protein includes a second binding partner capable of binding to the second binding tag, a fourth cleavable domain, and a fourth domain in this order from an N terminus to a C terminus,
the second protein and the fourth protein form a dimer via a binding between the second domain and the fourth domain,
the third protein and the fourth protein form a dimer via a binding between the third domain and the fourth domain,
the first binding tag and the first binding partner bind together, and
the second binding tag and the second binding partner bind together.

### Supplementary Note 82

The protein according to Supplementary Note 81, wherein the third cleavable domain and the fourth cleavable domain are the same cleavable domain.

### Supplementary Note 83

The protein according to Supplementary Note 81 or 82, wherein the third cleavable domain and/or the fourth cleavable domain includes a cleavable sequence of a self-cleaving peptide and/or a sequence capable of being cleaved by a protease or peptidase.

### Supplementary Note 84

The protein according to Supplementary Note 83, wherein the sequence capable of being cleaved by a protease is a thrombin cleavage sequence.

### Supplementary Note 85

The protein according to any one of Supplementary Notes 81 to 84, wherein the second binding tag and the second binding partner are a peptide tag and a peptide capable of spontaneously forming a covalent bond.

### Supplementary Note 86

The protein according to any one of Supplementary Notes 81 to 85, wherein a combination of the second binding tag and the second binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 87

The protein according to any one of Supplementary Notes 81 to 86, wherein the binding between the third domain and the fourth domain and/or the binding between the second domain and the fourth domain is a disulfide bond.

### Supplementary Note 88

The protein according to any one of Supplementary Notes 81 to 87,
wherein the first domain is a light chain of an antibody that binds to a first target,
the second domain is a heavy chain of the antibody that binds to the first target,
the third domain is a light chain of an antibody that binds to a second target, and
the fourth domain is a heavy chain of the antibody that binds to the second target.

### Supplementary Note 89

The protein according to Supplementary Note 88, wherein the antibody that binds to the first target and the antibody that binds to the second target recognize different epitopes.

### Supplementary Note 90

The protein according to Supplementary Note 88 or 89, wherein the antibody that binds to the first target and the antibody that binds to the second target recognize different antigens.

### Supplementary Note 91

The protein according to any one of Supplementary Notes 88 to 90, wherein the antibody that binds to the first target and the antibody that binds to the second target are IgG, IgA, IgE, IgD, or IgM.

### Supplementary Note 92

The protein according to Supplementary Note 91, wherein the IgG is IgG1, IgG2, IgG2a, IgG2b, IgG3, or IgG4.

### Supplementary Note 93

The protein according to any one of Supplementary Notes 81 to 92, wherein the first cleavable domain, the second cleavable domain, the third cleavable domain, and the fourth cleavable domain are the same cleavable domain.

### Supplementary Note 94

The protein according to any one of Supplementary Notes 81 to 93, wherein a combination of the first binding tag and the first binding partner and a combination of the second binding tag and the second binding partner are different from each other.

### Supplementary Note 95

The protein according to any one of Supplementary Notes 81 to 94,
wherein the second protein further includes a fifth cleavable domain and a third binding tag in this order at the C terminus,
the fourth protein further includes a sixth cleavable domain and a third binding partner capable of binding to the third binding tag in this order at the C terminus, and
the third binding tag and the third binding partner bind together.

### Supplementary Note 96

The protein according to Supplementary Note 95, wherein the fifth cleavable domain and the sixth cleavable domain are the same cleavable domain.

### Supplementary Note 97

The protein according to Supplementary Note 95 or 96, wherein the fifth cleavable domain and/or the sixth cleavable domain includes a cleavable sequence of a self-cleaving peptide and/or a sequence capable of being cleaved by a protease or peptidase.

### Supplementary Note 98

The protein according to Supplementary Note 97, wherein the sequence capable of being cleaved by a protease is a thrombin cleavage sequence.

### Supplementary Note 99

The protein according to any one of Supplementary Notes 95 to 98, wherein the third binding tag and the third binding partner are a peptide tag and a peptide capable of spontaneously forming a covalent bond.

### Supplementary Note 100

The protein according to any one of Supplementary Notes 95 to 99, wherein a combination of the third binding tag and the third binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

### Supplementary Note 101

The protein according to any one of Supplementary Notes 95 to 100, wherein the first cleavable domain, the second cleavable domain, the third cleavable domain, the fourth cleavable domain, the fifth cleavable domain, and the sixth cleavable domain are the same cleavable domain.

### Supplementary Note 102

The protein according to any one of Supplementary Notes 95 to 101, wherein a combination of the first binding tag and the first binding partner, a combination of the second binding tag and the second binding partner, and a combination of the third binding tag and the third binding partner are different from one another.

### <Nucleic Acid>

### Supplementary Note 103

A nucleic acid encoding the protein according to any one of Supplementary Notes 36 to 102.

### <Expression Vector>

### Supplementary Note 104

An expression vector including the nucleic acid according to Supplementary Note 103.

### <Transformant>

### Supplementary Note 105

A transformant including the nucleic acid according to Supplementary Note 103 and/or the expression vector according to Supplementary Note 104.

### <Method for Producing Protein>

### Supplementary Note 106

A method for producing a protein, including an expression step of expressing the nucleic acid according to Supplementary Note 103 and/or the expression vector according to Supplementary Note 104.

### Supplementary Note 107

The production method according to Supplementary Note 106,
wherein the expression step includes:
   a culturing step of culturing the transformant according to Supplementary Note 105; and
   an isolation step of isolating the protein according to any one of Supplementary Notes 36 to 102.

### INDUSTRIAL APPLICABILITY

As described above, with the present disclosure, it is possible to efficiently produce a heteromultimeric protein such as a bispecific antibody. Accordingly, the present disclosure is very useful in the fields of, for example, pharmaceuticals, pharmaceutical production, and the like.

### SEQUENCE LISTING

P22191WO.xml

1. A method for producing a heteromultimeric protein, comprising:
forming a first complex of two proteins by bringing the two proteins into contact with each other,
the two proteins including a first protein and a second protein,
the first protein including a first binding tag, a first cleavable domain, and a first domain in this order from an N terminus to a C terminus,
the second protein including a first binding partner capable of binding to the first binding tag, a second cleavable domain, and a second domain in this order from an N terminus to a C terminus,
the first protein and the second protein being capable of forming a dimer via a binding between the first domain and the second domain, and
the first complex being formed by the first binding tag and the first binding partner binding together and the first domain and the second domain binding together, and
generating a heterodimer composed of the first domain and the second domain by cleaving the first cleavable domain and the second cleavable domain in the first complex.

2. The production method according to claim 1,
wherein the first cleavable domain and the second cleavable domain are the same cleavable domain.

3-4. (canceled)

5. The production method according to claim 1,
wherein a combination of the first binding tag and the first binding partner is a combination of a binding tag and a binding partner selected from the group consisting of (1) to (3) below:
(1) an altered Streptococcus pyogenes surface protein (SpyCatcher) and a peptide tag (SpyTag) capable of binding to the SpyCatcher;
(2) an altered Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

6. (canceled)

7. The production method according to claim 1,
wherein the forming the first complex is forming a second complex of four proteins by bringing the four proteins into contact with one another,
the four proteins including the first protein, the second protein, a third protein, and a fourth protein,
the third protein including a second binding tag, a third cleavable domain, and a third domain in this order from an N terminus to a C terminus,
the fourth protein including a second binding partner capable of binding to the second binding tag, a fourth cleavable domain, and a fourth domain in this order from an N terminus to a C terminus,
the second protein and the fourth protein being capable of forming a dimer via a binding between the second domain and the fourth domain,
the third protein and the fourth protein being capable of forming a dimer via a binding between the third domain and the fourth domain, and
the second complex being formed by the first binding tag and the first binding partner binding together, the second binding tag and the second binding partner binding together, the first domain and the second domain binding together, the second domain and the fourth domain binding together, and the third domain and the fourth domain binding together, and
in the generating the heterodimer, a heterotetramer of the first domain, the second domain, the third domain, and the fourth domain is generated by cleaving the first cleavable domain, the second cleavable domain, the third cleavable domain, and the fourth cleavable domain in the second complex.

8. The production method according to claim 7,
wherein the third cleavable domain and the fourth cleavable domain are the same cleavable domain.

9-10. (canceled)

11. The production method according to claim 7,
wherein a combination of the second binding tag and the second binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

12. (canceled)

13. The production method according to claim 7,
wherein the first domain is a light chain of an antibody that binds to a first target,
the second domain is a heavy chain of the antibody that binds to the first target,
the third domain is a light chain of an antibody that binds to a second target, and
the fourth domain is a heavy chain of the antibody that binds to the second target.

14. The production method according to claim 13,
wherein the antibody that binds to the first target and the antibody that binds to the second target recognize different epitopes.

15. The production method according to claim 13,
wherein the antibody that binds to the first target and the antibody that binds to the second target recognize different antigens.

16. The production method according to claim 7,
wherein the first cleavable domain, the second cleavable domain, the third cleavable domain, and the fourth cleavable domain are the same cleavable domain.

17. (canceled)

18. The production method according to claim 7,
wherein the second protein further includes a fifth cleavable domain and a third binding tag in this order at the C terminus,
the fourth protein further includes a sixth cleavable domain and a third binding partner capable of binding to the third binding tag in this order at the C terminus,
in the forming the second complex, the third binding tag and the third binding partner also bind together, and
in the generating the heterotetramer, the fifth cleavable domain and the sixth cleavable domain in the second complex are cleaved.

19. The production method according to claim 18,
wherein the fifth cleavable domain and the sixth cleavable domain are the same cleavable domain.

20-21. (canceled)

22. The production method according to claim 18,
wherein a combination of the third binding tag and the third binding partner is a combination of a binding tag and a binding partner selected from the group consisting of (1) to (3) below:
(1) a Streptococcus pyogenes surface protein (SpyCatcher) and a peptide tag (SpyTag) capable of binding to the SpyCatcher;
(2) a Streptococcus pneumoniae protein (SnoopCatcher) and a peptide tag (SnoopTag) capable of binding to the SnoopCatcher; and
(3) an altered Clostridium perfringens protein Cpe0147₄₃₉₋₅₆₃ and a peptide tag Cpe0147₅₆₅₋₅₈₇ capable of binding to the Cpe0147₄₃₉₋₅₆₃.

23. The production method according to claim 18,
wherein the first cleavable domain, the second cleavable domain, the third cleavable domain, the fourth cleavable domain, the fifth cleavable domain, and the sixth cleavable domain are the same cleavable domain.

24-28. (canceled)

29. A protein comprising two proteins,
wherein the two proteins include a first protein and a second protein,
the first protein includes a first binding tag, a first cleavable domain, and a first domain in this order from an N terminus to a C terminus,
the second protein includes a first binding partner capable of binding to the first binding tag, a second cleavable domain, and a second domain in this order from an N terminus to a C terminus,
the first protein and the second protein form a dimer via a binding between the first domain and the second domain, and
the first binding tag and the first binding partner bind together.

30. The protein according to claim 29, further comprising a third protein and a fourth protein,
wherein the third protein includes a second binding tag, a third cleavable domain, and a third domain in this order from an N terminus to a C terminus,
the fourth protein includes a second binding partner capable of binding to the second binding tag, a fourth cleavable domain, and a fourth domain in this order from an N terminus to a C terminus,
the second protein and the fourth protein form a dimer via a binding between the second domain and the fourth domain,
the third protein and the fourth protein form a dimer via a binding between the third domain and the fourth domain,
the first binding tag and the first binding partner bind together, and
the second binding tag and the second binding partner bind together.

31. A nucleic acid encoding the protein according to claim 29.

32. An expression vector comprising the nucleic acid according to claim 31.

33. A transformant comprising the nucleic acid according to claim 31.

34. A method for producing a protein, comprising expressing the nucleic acid according to claim 31.
